(19) United States
(12) Patent Application Publication
(10) Pub. No.: US 2013/0058943 A1
(43) Pub. Date: Mar. 7, 2013

(54) BOVINE POLYCLONAL ANTIBODY SPECIFIC FOR HUMAN TNF

(71) Applicants: Barbara S. Fox, Wayland, MA (US); Eileen F. Bostwick, Waltham, MA (US); Daniel E. Tracey, Harvard, MA (US); Lisa D. Schlehuber, Arlington, MA (US); Michael S. Quesenberry, Douglas, MA (US)

(72) Inventors: Barbara S. Fox, Wayland, MA (US); Eileen F. Bostwick, Waltham, MA (US); Daniel E. Tracey, Harvard, MA (US); Lisa D. Schlehuber, Arlington, MA (US); Michael S. Quesenberry, Douglas, MA (US)

(21) Appl. No.: 13/564,566

(22) Filed: Aug. 1, 2012

Related U.S. Application Data
(63) Continuation-in-part of application No. 13/402,527, filed on Feb. 22, 2012.
(60) Provisional application No. 61/513,872, filed on Aug. 1, 2011.

(51) Int. Cl. C07K 16/24; C07K 1/36; A61P 1/00; A61K 39/395; A61P 29/00

(52) U.S. Cl. 424/139.1; 530/389.2; 530/387.9; 424/158.1; 424/157.1

(57) ABSTRACT

The invention provides a composition comprising polyclonal antibodies that specifically bind to with human tumor necrosis factor (hTNF) wherein the polyclonal antibodies are derived from the serum, milk or colostrum of a bovine animal that has been immunized hTNF or an immunogenic portion thereof. The antigenic specificity analysis of the anti-hTNF polyclonal antibodies of the invention is unique and has not been previously described for anti-hTNF polyclonal or monoclonal antibodies of the prior art.

### RELATED APPLICATION

This application is a continuation-in-part of U.S. application Ser. No. 13/402,527, filed on Feb. 22, 2012. This application claims the benefit of U.S. Provisional Application No. 61/513,872 filed on Aug. 1, 2011. The entire teachings of the above applications are incorporated herein by reference.

### GOVERNMENT SUPPORT

The invention was supported, in whole, or in part, by NIH grant numbers 1R43DE019735-01, 1R43DK083810-01A1, and 2R44DK083810-02 and by HHS contract HHSO100201100027C. The Government has certain rights in the invention.

### BACKGROUND OF THE INVENTION

Antibodies are an important class of pharmaceuticals. Antibodies specific for a target antigen have proven to be highly effective therapeutics in treating cancers and autoimmune disease, and their use has been of great benefit to afflicted patients. Antibodies are generally highly specific for a particular target and thus tend to have less off-target toxicity than is seen with small molecule therapeutics.

WO 2009/046168, WO 2009/020748 and US 20070184049 A1 describe the use of polyclonal antibodies derived from the milk of immunized mammals for use as therapeutics topically delivered to the digestive tract to target antigens that modulate the pathogenesis of one or more diseases. Colostrum and milk, particularly from bovine sources, are a uniquely safe source of polyclonal antibody for oral delivery to a human patient. There is already extensive human exposure to bovine immunoglobulin, as regular milk contains approximately 1.5 g/L IgG. However, milk and colostrum contain other components which on their own have therapeutic uses, but that may not be ideal in the context of treating certain diseases using polyclonal antibodies derived from a milk source. In addition to specific antibodies induced by immunization of the donor animal, milk and colostrum contain antibody with other specificities and many other biologically active non-immunoglobulin factors including, but not limited to proteins, peptides, and small molecules (reviewed in Korhnonen {Korhonen and Pihlanto, 2007, Curr Pharm Des, 13, 829-43} and Liang {Liang et al., 2011, Int J Environ Res Public Health, 8, 3764-76}).

Specific non-immunoglobulin components in milk and colostrum, many of which have biological activity either alone or in combination include lactoferrin, lactoperoxidase, alpha-lactalbumin, beta-lactoglobulin, transferrin, lysozyme, EGF, FGF, IGF-1, IGF-2, TGF-α, TGF-β1, TGF-β2, PDGF, VEGF, NGF, CTGF, insulin, protease, PRP, glutamine, polyamines, nucleotides, prolactin, somatostatin, oxytocin, luteinizing hormone-releasing hormone, TSH, thyroxine, calcitonin, estrogen, progesterone, IL-1b, TNF, IL-6, IL-10, IL-8, G-CSF, IFN-gamma, GM-CSF, C3, C4, mammary-derived growth factor II, human milk growth factor III; growth hormone and growth hormone releasing factor, casein, casein-derived peptides, Vitamins B1, B2, B6, B12, E, A, C, Folic Acid, pantothenic acid, beta-carotene, glycogen, retinoic acid, calcium, chromium, iron, magnesium, phosphorous, potassium, sodium, zinc, isoleucine, leucine, histidine, methionine, lysine, threonine, phenylalanine, valine, tryptophan, arginine, cysteine, glutamic acid, alanine, tyrosine, proline, aspartic acid, serine, β-2 microglobulin, haemopexin, haptoglobulin, orotic acid, peroxidase, and xanthine oxidase.

Colostrum is widely used as a nutritional supplement and has been studied as a therapeutic. {Khan et al., 2002, Aliment Pharmacol Ther, 16, 1917-22}. It has also been shown to be effective in animal models of colitis {Bodammer et al., 2011, J Nutr, 141, 1056-61}.

Many researchers have taken advantage of the therapeutic uses of such non-immunoglobulin components of colostrum and milk by concentrating one or more of the above-listed non-immunoglobulin components and depleting out other components such as immunoglobulin and casein. Potential therapeutic uses for such concentrated growth factors include the treatment of digestive ailments and the treatment of digestive inflammation. Colostrum has been considered as a beneficial treatment for a variety of intestinal ailments. Growth factors derived from milk or colostrum have been considered for their use in chemotherapy-induced mucositis. Methods for enriching for milk-derived growth factors and other bioactive components are known in the art. The art discloses compositions of bovine derived antibodies for oral administration of the treatment of diseases, particularly gastrointestinal diseases resulting from infection by a pathogen. However, the art does not contemplate the use of such antibodies in isolation from non-immunoglobulin components found in milk or colostrum, particularly when delivered orally. Indeed it was previously believed that such non-immunoglobulin components of colostrum stabilize the antibodies for oral administration.

It has not previously been appreciated that the presence of multiple active non-immunoglobulin factors in a pharmaceutical antibody product may be problematic. Some of the issues raised by the presence of non-immunoglobulin bioactives are listed here. First, levels of some of these non-immunoglobulin factors are affected by the health of the cow, by farm management practices, and by the stage of lactation during which collection occurred. For instance, in one survey of colostrum from 55 cows, {Kehoe et al., 2007, J Dairy Sci, 90, 4108-16} the average level of lactoferrin was 0.8 mg/ml but the range from individual cows was 0.1 mg/ml-2.2 mg/ml. This introduces a source of variability into the product which may make it difficult to achieve the consistency of manufacture required for a licensed biologic.

The variability in expression of these non-immunoglobulin factors is particularly challenging because it has not been possible to cleanly identify a single component or mixture of components that is responsible for the biological activity of colostrum. On the one hand, this makes it very difficult to achieve product uniformity. On the other hand, it makes it difficult to set specifications around the product.

Second, some of these non-immunoglobulin factors may act on the same pathways or disease processes that are being targeted by the specific antibodies in the therapeutic. This will make it difficult to evaluate the therapeutic benefit that results from administration of the specific antibody.

Third, some of these non-immunoglobulin factors may be associated with safety concerns, particularly when given to patients with gastrointestinal diseases. This is particularly true when the antibody product is intended to be administered chronically. For example, long-term exposure to growth factors may increase the risk of malignancy.

Thus there is a need to develop compositions and methods to permit the manufacture of a consistent antibody product that is free from potentially therapeutically confounding activities including the presence of non-immunoglobulin factor impurities.

Antibodies are generally highly specific for a particular target and thus tend to have less off-target toxicity than is seen with small molecule therapeutics. Monoclonal antibodies specific for tumor necrosis factor alpha, referred to herein as “TNF”, include therapeutic monoclonal antibodies known as REMICADE®, HUMIRA®, CIMZIA®, are effective in the treatment of inflammatory bowel disease. However, their use is associated with an increased risk of malignancy and an increased risk of serious infection (Bongartz et al., 2006, JAMA, 295, 2275-85), likely due to systemic immunosuppression. Therefore, there is a need to generate methods and pharmaceutical compositions of anti-TNF antibodies that are able to direct the antibody to the location where clinical benefit will be greatest while minimizing the activity of the antibody in other sites.

The use of anti-TNF antibody therapeutics is also frequently limited by the immunogenicity of the administered antibody. The induction of antibodies against the therapeutic agent is associated with loss of activity and with the potential for adverse infusion reactions. Immunogenicity is seen most clearly in cases where the antibody is derived from a non-human species. However, the presence of human anti-human antibody responses (HAHA) has also been described and is the cause of significant clinical concern (Ritter et al., 2001, Cancer Res, 61, 6851-9; Tracey et al., 2008, Pharmacol Ther, 117, 244-79). In the treatment of inflammatory bowel disease, patients treated with systemically administered anti-TNF antibodies frequently become non-responsive to antibody therapy due to the induction of neutralizing antibodies (Tracey et al., 2008, Pharmacol Ther, 117, 244-79). Thus, there is a need to generate methods and pharmaceutical compositions of antibody therapeutics that are able to minimize immunogenicity while maintaining efficacy and a need to generate methods and pharmaceutical compositions that can be used to treat patients who have become unresponsive to existing anti-TNF antibody therapeutics.

The use of a polyclonal antibody has potential clinical advantages compared with the use of a monoclonal antibody due to the presence of multiple reactivities in a polyclonal antibody against the antigenic target. There may be generated a polyclonal antibody which has reactivities against all or multiple epitopes on an antigenic target, such as human tumor necrosis factor (hTNF). Due to the polyclonal nature of the composition which contains many epitope specificities, the functional antibody density which can be achieved on the target antigen when using a polyclonal antibody is significantly higher than with a monoclonal antibody. This results in more efficient blocking or clearance of the target antigen. In addition, due to the polyclonal nature of the composition, several epitopes on the target antigen can be blocked at the same time, resulting in more efficient blocking of biological activity. Since biologically active TNF exists as a trimer displaying multiple copies of individual epitopes (Santora et al, 2001, Anal Biochem. 299:119-29), polyclonal antibodies are more effective at forming immune complexes by cross-linking trimeric TNF molecules via multiple repeating epitopes. Such immune complexes would facilitate more efficient clearance of biologically active TNF by the reticuloendothelial system.

In contrast to a monoclonal antibody, a polyclonal antibody preparation comprises a mixture of specificities, and therefore any single and individual, cross-reacting antibody of a particular specificity will be delivered at a very low concentration, thus reducing significantly the potential for harmful side-effects due to cross-reactivity. Any unwanted cross-reactivity of the polyclonal antibody preparation can be removed by further purification of the antibody composition. If a monoclonal antibody results in an unwanted cross-reactivity, it is inherent to the single antibody present and can of course not be removed without destroying the activity of the preparation.

In terms of the diminished potential for cross-reactivity, polyclonal antibodies will also be much less likely than monoclonal antibodies to induce a neutralizing anti-idiotype immune response, since each single epitope-specific idiotype of the administered polyclonal antibody preparation is present in a very low quantity or concentration, likely being below the threshold for generation of an anti-idiotype response.

Although polyclonal antibodies bind to multiple epitopes on the target antigen, a particular polyclonal antibody will not recognize all possible epitopes. Certain epitopes are immunogenic, while other epitopes are non-immunogenic. Certain immunogenic epitopes will have a greater degree of immunodominance than other immunogenic epitopes. The relative immunodominance of a particular immunogenic epitope is influenced by the degree of homology between the epitope and homologous epitopes naturally present in the host animal in which the polyclonal antibody is generated. The relative immunodominance is further influenced by the exposure of that epitope to the host immune response and can be manipulated by unfolding or otherwise denaturing the antigen prior to immunization, either intentionally or by interaction with a particular adjuvant. The relative immunodominance is further influenced by the particular immunization regimen that is used, as both the dose of immunogen and the number and timing of boosters have the potential to alter the relative recognition of particular epitopes by selection of particular antibody specificities through the process known as affinity maturation.

The specificity of the polyclonal antibody is important to its function. Some individual antibody molecules in the polyclonal antibody may bind to the target antigen at sites that do not interfere with biological activity. Other individual antibody molecules may bind to the target antigen at sites that interfere with some biological activities but not others. This is particularly true for antigens that have a complex mechanism of action. TNF, for example, is a trimeric molecule that is active in both a soluble and membrane bound form and binds to at least two separate classes of receptors. Therefore, it is important to functionally define the specificity of the polyclonal antibody.

The mechanism of action of therapeutic anti-TNF antibodies in vivo is not well understood, particularly with regard to the relative contributions of many possible mechanisms that have been defined in vitro and the specific TNF epitopes involved. Possible mechanisms of inhibition of the soluble form of TNF include blocking its binding to either or both of the classes of TNF receptors, TNFR1 and TNFR2, on a wide variety of cell types, as well as mechanisms of clearance of immune complexes. The membrane-bound form of TNF can act as a ligand for TNFR2 or as a ‘receptor’ to initiate reverse signaling mechanisms, so anti-TNF antibodies can act on membrane-bound TNF either by blocking the interaction with TNFR2 or by activating reverse signaling mechanisms. Therefore, in order to ensure reproducibility between lots of polyclonal antibody, particularly those used as clinical therapeutics, it is important to have assays that define the particular specificity of the polyclonal antibody.

One way to define the specificity of the polyclonal antibody is to evaluate the ability of the polyclonal antibody to bind to and/or to functionally inhibit homologous antigens, such as the same antigen from different species. The species specificity of the antibody provides a fingerprint of the particular balance of epitopes recognized by the polyclonal antibody, and is therefore a key element in defining and identifying a particular polyclonal antibody. Binding can be evaluated using immunoassays such as ELISAs or RIAs or using direct binding assays such as equilibrium dialysis or surface plasmon resonance using a BIAcore instrument. TNF neutralization function can be evaluated in assays for soluble TNF or membrane bound TNF such as by standard in vitro L929 assays.

Another way to define the specificity of the polyclonal antibody is to evaluate binding and inhibition of recombinant variants of hTNF, molecules related to TNF, such as other cytokines (e.g. lymphotoxin), and binding to peptide fragments of hTNF.

Another way to define specificity of the polyclonal antibody is by epitope mapping. Epitope mapping of a polyclonal antibody provides information on those portions of the target antigen which interact and bind with the polyclonal antibodies.

There is a need for new anti-TNF agents that have fewer potential side effects then the agents of the prior art when used in a clinical setting.

### SUMMARY OF THE INVENTION

In one embodiment, the invention provides a composition comprising polyclonal antibodies that specifically bind to hTNF derived from the serum, milk or colostrum of a bovine animal that has been immunized with human hTNF or an antigenic portion thereof. Such polyclonal antibodies are also referred to herein as “anti-hTNF polyclonal antibodies” or “bovine-derived anti-hTNF polyclonal antibodies”. The terms “specifically bind hTNF” or “is specific for hTNF” as used herein means that the polyclonal antibodies of the invention are capable of binding to hTNF.

The degrees of cross-reactivity with the TNF of various species effectively defines which epitopes the polyclonal antibodies predominantly bind in hTNF, since some epitopes are highly conserved and other epitopes differ among species. The epitope profile defined by cross-reactivity of TNF from different species could not be predicted from the sequence homologies of various species of TNF, nor from the adjuvants used in immunization; therefore the TNF species specificity profile of the anti-human TNF polyclonal antibodies is unexpected and defines the unique composition of the invention. In accordance with the invention, antigenic specificity analysis of the anti-hTNF polyclonal antibodies of the invention is unique and has not been previously described for anti-hTNF polyclonal or monoclonal antibodies of the prior art.

Due to the high degree of homology (>75%) of the primary structure of TNF across species from rodents to primates, it is expected that some of the antigenic epitopes on human TNF recognized by bovine polyclonal antibodies would be shared with TNF molecules from other species. However, the degrees of cross-reactivity of polyclonal antibodies with various species of TNF are representative of the unique antigenic specificity of the anti-hTNF polyclonal antibodies of the invention. Furthermore, the patterns of cross-reactivity, both for TNF-binding antibodies and for TNF-neutralizing antibodies, across various species of TNF are unique fingerprints of the antigenic specificity of the anti-hTNF polyclonal antibodies of the invention.

In a preferred embodiment, the polyclonal antibodies of the invention bind and neutralize human TNF. In one embodiment, the polyclonal antibodies also bind and/or neutralize TNF from a non-human primate selected from cynomolgus monkey TNF and Rhesus macaque TNF. In one embodiment, in addition to binding hTNF, the polyclonal antibodies also bind canine TNF. In one embodiment the polyclonal antibodies bind canine TNF to a greater degree than cynomolgus monkey and neutralize cynomolgus monkey TNF to a greater degree than canine TNF. In one embodiment, the polyclonal antibodies have less than about 2% and preferably less than about 1% cross-reactivity with murine TNF as compared to the cross reactivity of the antibodies with hTNF. In one embodiment the polyclonal antibodies have negligible activity in neutralizing murine TNF.

In one embodiment, the polyclonal antibodies bind at least one epitope on hTNF within approximately the amino acid positions selected from: amino acids 1-15 of SEQ ID NO: 1; amino acids 21-35 of SEQ ID NO: 1; amino acids 61-65 of SEQ ID NO: 1; amino acids 91-95 of SEQ ID NO: 1; amino acids 131-140 of SEQ ID NO: 1; and any combination thereof. In one embodiment the polyclonal antibodies of the invention bind to at least one epitope of hTNF wherein the hTNF epitope comprises an amino acid sequence selected from: SEQ ID NO: 2; SEQ ID NO: 3; SEQ ID NO: 4; SEQ ID NO: 5; and SEQ ID NO: 6.

In one embodiment, the anti-hTNF polyclonal antibodies of the invention induce apoptosis in peripheral blood mononuclear cells (PBMCs) expressing transmembrane TNF.

In one embodiment, the potency of the polyclonal antibodies specific for hTNF is at least 1 mg/ml when tested by ELISA (where potency is defined as the concentration of antibody generating a half-maximal response and where the concentration is defined by protein concentration in the BCA assay). In one embodiment the polyclonal antibodies inhibit hTNF in a cytotoxicity assay and preferably the potency of hTNF inhibition in the assay when calculated as an EC₅₀ is about 0.1 mg/ml, more preferably 0.03 mg/ml or more (where EC₅₀ is defined as the concentration of antibody generating a half-maximal inhibition of hTNF and where the concentration is defined by protein concentration in the BCA assay).

In one embodiment, the polyclonal antibodies of the invention possess a combination of any two or more of the above featured embodiments.

The invention also provides pharmaceutical compositions and methods for using the anti-hTNF polyclonal antibody pharmaceutical compositions of the invention in the treatment of diseases wherein TNF is implicated in the pathology of the diseases. In one preferred embodiment, the pharmaceutical compositions of the invention are topically delivered to a diseased area in the digestive tract such as oral or rectal delivery to the digestive tract.

In one preferred embodiment, pharmaceutical compositions comprising anti-hTNF polyclonal antibody compositions of the invention useful in the treatment of inflammatory diseases including inflammatory diseases of the digestive tract such as inflammatory bowel disease including ulcerative colitis and Crohn's disease.

In one embodiment, the pharmaceutical compositions comprising anti-hTNF polyclonal antibody compositions of the invention are useful in the treatment of oral or gastrointestinal mucositis including mucositis caused by radiation therapy or chemotherapy.

In one embodiment, the pharmaceutical compositions comprising anti-hTNF polyclonal compositions of the invention are useful in the treatment of inflammation and damage to the digestive tract resulting from the exposure to radiation including therapeutic exposure to radiation and non-therapeutic exposure to radiation including gastrointestinal acute radiation syndrome (GI-ARS).

In one embodiment, the invention comprises administering the polyclonal antibodies of the invention to a non-human animal for gathering preclinical or clinical data. The non-human animal may be healthy (e.g. toxicology studies) or may be suffering from a disorder to be treated with the hTNF polyclonal antibodies of the invention, such as a non-human animal model for the target disease. In one embodiment the non-human mammal being tested is an animal model for mucositis or inflammatory bowel disease such as those animal models described in Bowen et al., J. Support. Oncol (2011) 9:161-168; Mizoguchi and Mizoguchi, (2008) J. of Gastroenteraol (2008) 43:1-17; and Watkins et al. (1997) Gut 40:628-633. In one embodiment the non-human mammal being tested is an animal model for gastrointestinal acute radiation syndrome (GI-ARS) such as one of the animal model described in Williams et al., Radiation Research Society (2010) 173:557-578. In one embodiment the non-human anima is an animal which expresses a homologue of TNF that is cross reactive with the hTNF polyclonal antibodies of the invention such as a dog, monkey mini pig or guinea pig.

The invention also provides methods for preparing a composition comprising polyclonal antibodies that bind specifically to hTNF comprising the steps of: inoculating a bovine animal with hTNF or an antigenic portion thereof and an adjuvant, preferably wherein in the adjuvant is Quil A, Montanide ISA 201 VG, EMULSIGEN®-D or EMULSIGEN® BCL; and recovering serum, milk or colostrum from the bovine animal after the animal has had an immune response to the hTNF.

In one embodiment, the composition of the invention is depleted of non-immunoglobulin factors. In one embodiment, the biological source is milk or colostrum. In one preferred embodiment the biological source is milk or colostrum from an animal immunized with the target antigen or immunogenic portion thereof. In one embodiment, the compositions are depleted of lactoferrin. In one embodiment, the compositions are depleted of low molecular weight growth factors. In one embodiment, the compositions are depleted of non-immunoglobulin factors and are further depleted of immunoglobulins that are not specific for the target antigen.

The polyclonal anti-hTNF antibodies of the invention are as potent as monoclonal anti-hTNF antibodies of the prior art as tested in standard cytotoxicity bioassays for neutralization of hTNF but are expected to have fewer side effects when used in a clinical setting. Characterization of the anti-hTNF polyclonal antibodies of the invention and the processes for making and using them and optionally purifying them are described herein.

### BRIEF DESCRIPTION OF THE FIGURES

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a line graph showing anti-human TNF ELISA data. ELISA plates were coated with rhTNF and bovine anti-TNF serum (Serum-470) samples in a 3 fold-dilution series were added to the plates. Colorimetric analysis was performed and optical densities were determined at 450 nm. Shown are curves of the antibodies present in the antiserum to human TNF generated with four different adjuvants.

FIG. 2 is a line graph showing anti-bovine Ig ELISA data. ELISA plates were coated with anti-bovine IgG. Pools of Serum-470 were serially diluted and added to the plates and washed. Binding was detected using peroxidase-conjugated anti-bovine IgG antibody. Shown is the binding curve of the antibodies present in the antiserum to human TNF generated with four different adjuvants.

FIG. 3 is a bar graph showing the relative ELISA titers of Serum-470 (Quil A adjuvant) for TNF from different animal species.

FIG. 4 is a bar graph showing the relative ELISA titers and L929 IC50s of Serum-470 (Quil A adjuvant) for TNF from different animal species.

FIG. 5 is a line graph showing anti-bovine Ig ELISA data.

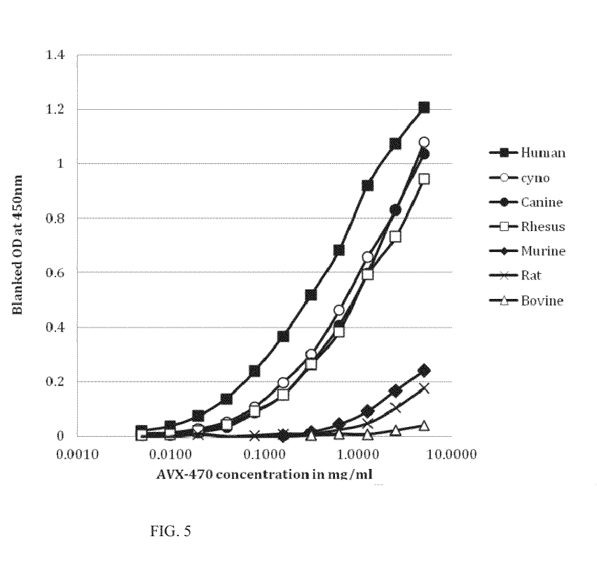

FIG. 6 is a line graph showing the L929 IC50s for TNF from different animal species.

FIG. 7 is line graph showing the affinity for affinity purified human AVX-470 (AVX-470A) for TNF as measured by a competition ELISA.

FIG. 8 is a line graph showing the potency of affinity purified human AVX-470 (AVX-470A) as measured by ELISA.

FIG. 9 is a line graph showing the potency of affinity purified AVX-470 (AVX-470A) as evaluated by neutralization in a standard in vitro L929 assay.

FIG. 10 shows the readout of a FACS analysis showing the induction of apoptosis of human cells treated with AVX-470 or infliximab.

### DETAILED DESCRIPTION OF THE INVENTION

The term “immunoglobulin (Ig) and their plural forms, as used herein refer to a polypeptide comprising a framework region from an immunoglobulin gene or fragments thereof that specifically binds and recognizes an antigen. Immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD (not found in bovines) and IgE, respectively. Typically, the antigen-binding region of an immunoglobulin will be most critical in specificity and affinity of binding to a target receptor. An exemplary immunoglobulin structural unit comprises a tetramer and is also referred to herein as an “antibody” or “antibodies” and include polyclonal antibodies. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Immunoglobulins exist, e.g., as intact antibodies or as a number of well-characterized antibody fragments produced by degradation with various peptidases. (e.g. Fab, F(ab′)₂, Fab′, Fc). Immunoglobulins also exist, for example, as fragments that may be present in a biological source such as milk or colostrum that are the result of natural degradation or degradation associated with processing of the milk or colostrum. As used herein the term immunoglobulins includes polypeptides that are associated with immunoglobulins such as the secretory component and J chain components associated with IgA and IgM. Therefore, as used herein the term immunoglobulin (Ig) compositions refers to compositions of intact antibodies (including polyclonal antibodies) or fragments thereof or protein components associated therewith derived from all immunoglobulin isotypes.

The terms “polyclonal antibody” or “polyclonal antibodies” as used herein refer to a composition comprising different antibody molecules which are capable of binding to or reacting with several different specific antigenic determinants (also referred to herein as “epitopes”) on the same or on different antigens. The variability in antigen specificity of a polyclonal antibody is located in the variable regions of the individual antibody molecules constituting the polyclonal antibody and in the particular mixture of antibody molecules that constitute the polyclonal antibody. Preferably, compositions comprising the polyclonal antibody of the invention are prepared by immunization of an animal with the target antigen or portions thereof as specified below and are derived from the blood, milk or colostrum obtained from the immunized animal.

Compositions comprising polyclonal antibodies derived from the serum (blood), milk, or colostrum of immunized animals typically include antibodies that are not specific for the immunogen in addition to antibodies specific for the target antigen. In accordance with one preferred embodiment, the anti-hTNF polyclonal antibody compositions of the invention comprise at least 0.3% or more of antibodies that specifically neutralize hTNF.

Other non-immunoglobulin factors may also be present in polyclonal antibody compositions of the invention derived from the blood, milk or colostrum of animals. Polyclonal antibodies specific for the target antigen (e.g., hTNF) may be further purified from the polyclonal antibody preparation or the polyclonal antibody preparation may be used without further purification. However, in a preferred embodiment, the polyclonal antibodies of the invention have been substantially depleted of non-immunoglobulin factors as is described herein.

The terms “monoclonal antibody” or “monoclonal antibodies” as used herein refer to a preparation of antibodies of single molecular composition. A monoclonal antibody composition displays a single binding specificity and affinity for a particular epitope of a target antigen. A mixture of monoclonal antibodies is not as such considered a polyclonal antibody. However, if a mixture of monoclonal antibodies provides the same unique characteristics of the polyclonal antibodies of the present invention, such monoclonal antibodies are considered an equivalent of the polyclonal antibodies of the invention.

An “epitope” is the portion of a molecule that is bound by an antibody. An epitope is also referred to as a determinant or antigenic determinant. Polyclonal antibodies binding to different epitopes on the same antigen can have varying effects on the activity of the antigen they bind depending on the location of the epitope. An antibody binding to an epitope in an active side of the antigen may block the function of the antigen completely, whereas another polyclonal antibody binding at a different epitope may have no or little effect on the activity of the antigen alone. Such antibodies may however still activate complement or other effector functions and thereby result in the elimination of the antigen and may result in synergistic effects of the polyclonal antibodies binding to different epitopes on the same antigen.

The “immunogenic portion” or “immunogenic fragment” of an antigen or immunogen is any portion of the antigen immunogen that is capable of inducing an immune response in the host animal being immunized with the antigen or immunogen and that preferably causes the animal to generate polyclonal antibodies against the target antigen. In a preferred embodiment the immunogen is hTNF or an immunogenic fragment thereof.

For the purposes of the invention, the “digestive tract” consists of the mouth, pharynx, esophagus, stomach, small intestine (duodenum, jejunum, ileum), large intestine (cecum, colon, rectum) and anus. For the purposes of the invention, the “oral cavity” is understood to include the mouth, the pharynx and the esophagus. For the purposes of the invention, the “gastrointestinal tract”, or “GI tract” is understood to include the stomach, small intestine (duodenum, jejunum, ileum), large intestine (cecum, colon, rectum) and anus.

Human tumor necrosis factor (hTNF) is a cytokine that is implicated in the pathogenesis of many diseases including inflammatory diseases such as inflammatory bowel disease which includes Crohn's disease and ulcerative colitis. Inhibition of TNF has also been shown to treat mucositis including mucositis resulting from chemotherapy or radiation therapy, and damage due to exposure to radiation from therapeutic sources such as radiation therapy for treating cancer and other diseases and non-therapeutic sources including GI acute radiation syndrome (GI-ARS) (WO 2009/046168).

In humans and animal species, TNF is released from cells as mature/soluble TNF (sTNF), a homotrimer of 17-kDa monomers, after being enzymatically cleaved from its cell surface-bound precursor, transmembrane TNF (tmTNF), a homotrimer of 26 kDa monomers. Recombinant human TNF and recombinant TNF from various animal species are genetically-derived homologs of sTNF expressed by cDNA-transfected bacterial cells and purified to homogeneity. The biological functions of TNF are initiated by binding of TNF trimers to either of two distinct TNF receptors, TNFR1 and TNFR2 on the surface of a wide variety of cell types. It would be expected that antibodies directed against epitopes on TNF that are on or near the receptor-binding amino acids could block the binding of TNF to its receptors and neutralize its biological activities. In addition, some anti-TNF antibodies may bind to epitopes on TNF that do not interfere with receptor binding and do not neutralize TNF activity. Human TNF monomers are 157 amino acids long and are represented herein by amino acids 1-157 of SEQ ID NO: 1, but may also be represented by amino acids 76-233 of the precursor form, and are not glycosylated.

The term “non-immunoglobulin factors” as used herein includes non-immunoglobulin proteins and peptides, non-immunoglobulin macromolecules and small molecules. Antibodies that are present in the biological source such as colostrum, milk or serum that are not specific for the target antigen are referred to herein as “non-specific antibodies”. The term “target antigen” refers to the antigen to which the polyclonal antibodies of a composition are intended to bind.

As is understood in the art, the target antigen is an antigen that is present in a patient who will ultimately be treated with the polyclonal antibody compositions of the invention that are specific to the target antigen. As such the polyclonal antibodies in accordance with the invention will bind the target antigen when administered to the patient. For example, for a polyclonal antibody specific for TNF, the target antigen is preferably human TNF (also referred to herein as “hTNF”) when the patient is a human patient.

In a preferred embodiment, a composition comprising the polyclonal antibodies specific for a hTNF that are isolated from the milk or colostrum of a bovine, preferably an immunized cow. In one embodiment the polyclonal antibodies are bovine IgG antibodies. In one embodiment, the polyclonal antibodies are bovine antibodies of mixed Ig isotypes present in milk or colostrum including IgA, IgM and IgG.

Bovine colostrum (early milk) is a preferred source of polyclonal antibody compositions for this invention. In cows, antibody does not cross the placenta, and thus all passive immunity is transferred to the newborn calf through the colostrum. As a result, cows secrete a large bolus of antibody into the colostrum immediately after parturition and approximately 50% of the protein in colostrum is immunoglobulin. In the first 4 hours after birth, immunoglobulin concentrations of 50 mg/ml are typically found in the colostrum (Butler and Kehrli, 2005, Mucosal Immunology, 1763-1793), dropping to 25-30 mg/ml 24 hours later (Ontsouka et al., 2003, J Dairy Sci, 86, 2005-11). As used herein the term “colostrum” refers to the lacteal secretions produced by the cow within the first 3 to 4 days after parturition. In some instances it will be specified that colostrum is isolated from a particular time frame after parturition (e.g. first milking colostrum, first day colostrum or colostrum from the first 3 to 4 days after parturition).

General methods of producing polyclonal antibodies that specifically to target antigens of the invention are known to those of skill in the art (see, e.g., Coligan, Current Protocols in Immunology (1991); Harlow & Lane, Antibodies, A Laboratory Manual (1988); Goding, Monoclonal Antibodies: Principles and Practice (2d ed. 1986); and Kohler & Milstein, Nature 256: 495-497 (1975). Such techniques include preparation of polyclonal antibodies by immunizing suitable animals (see, e.g., Huse et al., Science 246: 1275-1281 (1989); Ward et al., Nature 341: 544-546 (1989)). The present invention provides optimized methods for obtaining the polyclonal antibodies of the present invention and such methods are described herein.

A number of immunogens comprising hTNF or antigenic or immunogenic fragments or portions thereof may be used to produce antibodies specifically reactive with hTNF. For example, an antigenic or immunogenic fragment or protein portion of hTNF can be isolated from appropriate sources such as tissue cultures using known procedures. In a preferred embodiment, the immunogen is recombinant human TNF (rhTNF). Recombinant hTNF can be expressed in eukaryotic or prokaryotic cells and purified as is known in the art. Alternatively, a synthetic peptide derived from rhTNF can be used as an immunogen. The synthetic peptide may be conjugated to a carrier protein prior to immunization. Naturally occurring hTNF may also be used either in pure or impure form. The product is then injected into an animal capable of producing antibodies, preferably a bovine animal. Animals may also be immunized with cells that have been transfected with hTNF or may be immunized with DNA encoding hTNF.

In a preferred embodiment, the immunogen used for inoculation of the animal also includes an adjuvant to enhance the antibody response in the animal. The choice of the appropriate adjuvant is very important as adjuvants have the capability of influencing titer, isotype, avidity and properties of cell mediated immunity and as is demonstrated in the Examples herein, the antigenic species specificity profile of the anti-hTNF polyclonal antibodies of the invention. Adjuvants include but are not limited to water-in-oil emulsions such as Complete Freund's Adjuvant (CFA), Incomplete Freund's Adjuvant (IFA), and Montanide ISA 201 VG (incomplete seppic adjuvant, water-in-oil-in-water double emulsion) available from Seppic, Paris, France; Montanide ISA-25; oil-in-water emulsions such as EMULSIGEN®-D and EMULSIGEN®-BCL available from MVP Laboratories, Omaha Nebr.; aluminum salt adjuvants; Gerbu Adjuvants (GERBU Biochemicals GmbH, Gaiberg, Germany) based on the immunomodulator GMDP, a glycopeptide from the cell wall of L. bulgaricus; and saponins such as Quil A.

In one preferred embodiment the invention provides a method for preparing a composition comprising polyclonal antibodies that bind specifically to hTNF comprising the steps of: inoculating a bovine animal with hTNF and an adjuvant, preferably wherein in the adjuvant is Quil A, Montanide ISA 201 VG, EMULSIGEN®-D or EMULSIGEN® BCL; and recovering serum, milk, and/or colostrum from the bovine animal after the animal has had an immune response to the hTNF. In another preferred embodiment, the adjuvant is Montanide ISA-25.

In one embodiment, the animals receive three immunizations with the hTNF antigen and adjuvant combination, spaced 2-3 weeks apart. In one embodiment, the animals receive four immunizations with the hTNF antigen and adjuvant combination, spaced 2-3 weeks apart.

In one embodiment, the invention provides a composition of polyclonal antibodies that both bind and/or neutralize TNF from a species that is suitable for conducting safety toxicology studies. In the development of pharmaceutical drug products, it is necessary to test a novel drug substance or drug product in animals prior to the initiation of human clinical trials. In the development of some pharmaceutical drug products, such as those for the treatment of GI Acute Radiation Syndrome, it is not possible to evaluate efficacy in human clinical trials and approval to market the drug is based on efficacy studies in animal models. It is preferred that the drug substance or drug product displays relevant reactivity in the animal species. Cynomolgus monkeys and rhesus macaques are two preferred species. Dogs and pigs are other preferred species.

As used herein a polyclonal antibody of the invention that “neutralizes TNF activity”, refers to an antibody whose binding to TNF results in inhibition of the biological activity of TNF. This inhibition of the biological activity of TNF can be assessed by measuring one or more indicators of TNF biological activity, such as TNF-induced cytotoxicity (either in vitro or in vivo). These indicators of TNF biological activity can be assessed by one or more of several standard in vitro or in vivo assays known in the art and described in the examples.

Preferably, the ability of an antibody to neutralize TNF activity is assessed by inhibition of TNF-induced cytotoxicity of L929 cells as described in the examples. In one preferred embodiment, the polyclonal antibodies of the invention neutralize human TNF cytotoxicity in a standard in vitro L929 assay with a Ki of 4.0 pM or less.

The inhibition constant, Kᵢ, is a measure of the potency of an inhibitor. The Kᵢ for antibody inhibition of a ligand can be calculated from IC₅₀ values of an antibody at different ligand concentrations using an adaptation of the Cheng-Prusoff equation, originally developed to measure kinetic parameters of enzyme inhibitors (Cheng and Prusoff, (1973) Biochem Pharmacol 22: 3099-108).

### Cheng-Prusoff Equation:

Kᵢ=IC₅₀/[1+(A/EC₅₀)];

where:
IC₅₀=the dilution of serum needed to reduce TNF activity by 50%
A=the concentration of TNF used in the assay (usually the EC₉₀)
EC₅₀=the concentration of TNF needed to inhibit the growth of L929 cells by 50%.

In one preferred embodiment, the polyclonal antibodies of the invention neutralize human TNF cytotoxicity in a standard in vitro L929 assay with an EC50 of at least about 0.03 mg/ml.

In one embodiment, the invention provides polyclonal antibodies that also bind and neutralize TNF from at least one non-human primate selected from cynomolgus monkey or Rhesus macaque. For example the polyclonal antibodies of the invention bind cynomolgus TNF and Rhesus TNF at an EC50 that is within 2-fold of the EC50 with hTNF (see Example 27).

In one embodiment, the invention provides polyclonal antibodies that also bind canine TNF. In one embodiment the polyclonal antibodies neutralize cynomolgus monkey TNF to a greater extent than neutralization of canine TNF. In one embodiment, the polyclonal antibodies bind canine TNF to a greater extend than cynomolgus monkey TNF.

In one embodiment, the method provides polyclonal antibodies that have less than about 2% and preferably less than about 1% cross reactivity with murine TNF. This low cross reactivity with murine TNF is surprising given that the high level of amino acid identity between the human TNF and mouse TNF would give rise to the expectation that a population of polyclonal antibodies raised against human TNF would also include polyclonal antibodies specific for conserved epitopes on both the human and mouse forms of TNF that are not shared by the host species' (bovine) TNF. This surprising characteristic of the polyclonal antibodies partially defines the unique specificity and composition of the invention.

The present inventors have further discovered that the antigenic species specificity profile of polyclonal antibodies can be modulated during the process of preparing the polyclonal antibodies such as by selection of the adjuvant with which the target antigen is used to inoculate the bovine animal, the dose of immunogen, type of immunogen (e.g., fragment or full protein), and/or the immunization schedule. This is useful to further define the factors which contribute to the unique antigenic specificity and composition of the invention.

In one embodiment the ability of a polyclonal antibody of the invention to neutralize TNF activity is assessed by the antibody's ability to induce apoptosis in immune effector cells such as activated lymphocytes in cells expressing transmembrane TNF (Van den Brande et al. (2003) Gastroenterology 124:1774-1785). This can be assessed by an assay for apoptosis in relevant cells such as human PBMCs that express transmembrane TNF as described in Example 31.

The polyclonal antibodies of the invention preferably do not specifically bind to other cytokines such as lymphotoxin (LTα/TNFβ), IL-1α, IL-1β, IL-2, IL-4, IL-6, IL-8, IFNγ and TGFβ.

In one embodiment the titer of the polyclonal antibodies specific for hTNF is at least 100 when tested by ELISA. In one embodiment the titer of the polyclonal antibodies specific for hTNF is at least 300 when tested by ELISA. In one embodiment the EC50 of the polyclonal antibodies specific for hTNF is at least 1 mg/ml when tested by ELISA. In one embodiment the EC50 of the polyclonal antibodies specific for hTNF is at least 0.3 mg/ml when tested by ELISA.

In addition to polyclonal antibodies specific to the target antigen induced by immunization of the donor animal, milk and colostrum contain antibody with other specificities (referred to here as “non-specific immunoglobulins”) and many other proteins, peptides, and small molecules (referred to here as “non-immunoglobulin factors”). These non-immunoglobulin factors have a variety of biological activities and have generally been thought to be either benign or beneficial.

In one aspect of this invention, non-immunoglobulin factors are depleted from polyclonal antibody compositions of the invention during the manufacturing process. This depletion may be done by absorption of the impurities or the immunoglobulin onto affinity columns. Alternatively, this depletion can be performed using size exclusion chromatography or similar techniques. Alternatively, this depletion can be performed using ultrafiltration/diafiltration or similar techniques. Alternatively, this depletion can be performed by absorption of the impurities or the immunoglobulin onto ion exchange columns. A combination of the above-described methods for purifying and isolating immunoglobulins in accordance with the invention may be used.

In one aspect of this invention, the levels of specific non-immunoglobulin factors are monitored during in-process testing and as part of release testing of compositions comprising polyclonal antibodies directed to specific target antigens. In one embodiment, levels of all non-immunoglobulin factors are reduced at least 5 fold below the average levels in colostrum. In one embodiment, levels of all non-immunoglobulin factors are reduced at least 10 fold below the average levels in colostrum. In one embodiment, the polyclonal compositions of the invention are substantially free of non-immunoglobulin factors.

In one preferred embodiment, the non-immunoglobulin factor depleted from polyclonal antibody compositions of the invention is lactoferrin. In one preferred embodiment, the non-immunoglobulin factors depleted from polyclonal antibody compositions of the invention are one or more specific growth factors. In one embodiment, one or more specific growth factors are depleted at least 5-fold and preferably at least 10-fold below their natural levels in colostrum and preferably compositions of the invention are substantially free of growth factors.

Growth factors include but are not limited to insulin-like growth factor-1 (IGF-1), insulin-like growth factor-2 (IGF-2), epidermal growth factor (EGF), nerve growth factor (NGF), fibroblast growth factor (FGF), transforming growth factor-alpha (TGF-α), transforming growth factor-beta (TGF-β), platelet-derived growth factor (PDGF), vascular endothelial growth factor (VEGF), connective tissue growth factor (CTGF), growth hormone and insulin.

Table 1 provides data showing general levels of various non-immunoglobulin factors naturally found in milk and colostrum (Ontsouka et al., J. Dairy Sci. 86:2005-2011).

TABLE 1

| Factor | Colostrum (day 2) | Milk |
|---|---|---|
| IGF-1 (ug/ml) | 103 ± 21 | 4 ± 1 |
| Insulin (ug/ml) | 4.55 ± 1.04 | 0.37 ± 0.02 |
| Prolactin (ug/ml) | 120 ± 16 | 15.4 ± 1.0 |
| TNF-alpha (ug/ml) | 5.0 ± 0.6 | 1.8 ± 0.2 |
| Gamma- | 137 ± 9 | 24 ± 8 |
| glutamyltransferase | | |
| (ukat/L) | | |

Table 2 provides additional data showing general levels of various non-immunoglobulin factors naturally found in milk and colostrum (Su, C. K., and B. H. Chiang (2003) J Dairy Sci., 86:1639-1645).

TABLE 2

| Factor | Colostrum | Milk |
|---|---|---|
| Lactoferrin (mg/ml) | 1.0 | Negligible |
| BSA (mg/ml) | 1.0 | 0.4 |
| Beta-lactoglobulin (mg/ml) | 6.0 | 3.2 |
| Alpha-lactalbumin | 1.1 | 1.1 |

Table 3 provides data showing normal levels of various non-immunoglobulin factors found in milk and colostrum (Playford et al., 2000, Am. J. Clin. Nutr. 72:5-14).

TABLE 3

| Factor | Colostrum | Milk |
|---|---|---|
| TGF-beta (ug/ml) | 20-40 | 1-2 |
| IGF-1 (ug/ml) | 0.5 | 0.01 |

Non-immunoglobulin factors including growth factors that may be depleted from polyclonal antibody compositions of the invention derived from milk or colostrum in accordance with the invention include, but are not limited to those listed in Table 4.

TABLE 4

| Non-Immunoglobulin Factors | Examples |
|---|---|
| Growth Factors | EGF, FGF, IGF-1 IGF-2, TGF-α, TGF-β, |
| | PDGF, VEGF, NGF, CTGF, Growth |
| | Hormone, Insulin |
| Immunomodulators | Lactoferrin, Transferrin, Protease, PRP, |
| | IL-6, IL-8, IL-10, IF-γ, Lymphokines, |
| | Lysozyme, C3, C4, TNF specific to the |
| | host animal |
| Vitamins and | Vitamins B1, B2, B6, B12, E, A, C, Folic |
| Other Nutrients | Acid, Panthothenic Acid, Beta-carotene, |
| | Glycogen, Retinoic Acid |
| Minerals | Calcium, Chromium, Iron, Magnesium, |
| | Phosphorous, Potassium, Sodium, Zinc |
| Essential | Isoleucine, Leucine, Histidine, Methionine, |
| Amino Acids | Lysine, Threonine, Phenylalanine, Valine, |
| | Tryptophan |
| Nonessential | Arginine, Cysteine, Glutamic Acid, Alanine, |
| Amino Acids | Tyrosine, Glycine, Proline, Aspartic Acid, |
| | Serine |
| Additional | β-2 microglobulin, Haemopexin, |
| Factors | Haptoglobulin, Lactoperoxidase, |
| | Orotic Acid, Peroxidase, |
| | Xanthine Oxidase, Glycoproteins |

Key:
(−) = Negative regulation,
TGF = Transforming Growth Factor,
MCP = Macrophage Chemoattractant Protein,
MIP = Macrophage Inflammatory Protein,
GRO = Growth-Related Oncogene,
IL = Interleukin,
VEGF = Vascular Endothelial Growth Factor,
PLGF = Placenta Growth Factor,
FGF = Fibroblast Growth Factor,
HGF = Hepatocyte Growth Factor,
Cyr61 = Cysteine-Rich 61,
GM-CSF = Granulocyte-Macrophage Colony Stimulating Factor,
IP = Interferon-γ-Inducible Protein-10,
PDGF = Platelet-Derived Growth Factor,
CTGF = Connective Tissue Growth Factor,
IGF = Insulin-like Growth Factor,
NGF = Nerve Growth Factor,
EGF = Epidermal growth Factor,
HB-EGF = Heparin-Binding Epidermal Growth Factor,
NDF = Neu Differentiation Factors,
BMP = Bone Morphogenetic Proteins,
Ig = Immunoglobulin,
PRP = Proline-Rich Polypeptide,
C = Complement,
IF = Interferon-γ.

A polyclonal antibody composition of the invention that has been depleted of non-immunoglobulin factors are sometimes referred to herein as a “non-Ig factor-depleted polyclonal antibody compositions”. Such non-Ig factor-depleted polyclonal antibody compositions of the invention are suitable for use in the treatment of disease wherein the pathogenesis of the disease is modulated by a target antigen to which the polyclonal antibodies are directed. Such treatment also includes the mitigation of potential side effects associated with the use of polyclonal antibody compositions derived from a biological source in the treatment of disease whether the treatment is for acute disease or chronic disease.

The non-Ig factor-depleted polyclonal antibody compositions of the invention may be further processed to enrich for the presence of polyclonal antibodies specific for the target antigen wherein non-specific immunoglobulins have been selectively depleted or removed from the polyclonal antibody composition. Numerous techniques are known to those in the art for enriching polyclonal antibodies for antibodies to specific targets antigens. In one embodiment at least 60%, preferably at least 70%, preferably at least 80%, preferably at least 90%, and preferably at least 95% of the immunoglobulins present in a composition of the invention are polyclonal antibodies specific for a target antigen. In one embodiment, polyclonal antibody compositions are enriched for antibodies that bind to the target antigen such that the composition is substantially free of non-specific immunoglobulins. Non-Ig factor-depleted polyclonal antibody compositions that have been enriched for binding to a target antigen are sometimes referred to herein as “enriched non-Ig factor-depleted polyclonal antibody compositions.” In one embodiment, the present invention comprises polyclonal compositions wherein non-specific antigens are depleted and non-immunoglobulin factors are optionally depleted.

In a preferred embodiment, the invention provides a composition comprising isolated and purified immunoglobulin derived from the colostrum of a bovine that has been immunized with all or a portion of a target antigen wherein the composition comprises polyclonal antibodies capable of binding the target antigen and/or neutralizing the target antigen and/or modifying the function of the target antigen in standard assays as are known in the art. Such assays include but are not limited to ELISA, radioimmunoassay, immunodiffusion, flow cytometry, Western blotting, agglutination, immunoelectrophoresis, surface plasmon resonance, and assays based on neutralization or modulation of the function of the target antigen, such as neutralization of TNF in the L929 cell-based assay. In one embodiment, the composition is at least 90% immunoglobulin as measured by reducing SDS PAGE/densitometry. In a preferred embodiment, the composition is at least 95%, preferably at least 97%, preferably at least 98% and preferably at least 99% immunoglobulin as measured by reducing SDS-PAGE/densitometry.

In one embodiment at least one of lactoferrin (LF), alpha-lactalbumin (a-Lac), beta-lactoglobulin (b-Lac), lactoperoxidase (LPO) and insulin-like growth factor-1 (IGF-1) is depleted at least 10 fold below its normal level in colostrum.

In one preferred embodiment, lactoferrin is present in the immunoglobulin composition derived from the colostrum of a bovine at a level of no more than about 10 mg per gram of total protein present in the composition wherein the total protein content of the composition is measured by bicinchonic acid (BCA) assay (Smith, P. K., et al., Measurement of protein using bicinchoninic acid. Anal. Biochem. 150, 76-85, (1985)) and the level of lactoferrin is measured by ELISA. More preferably the level of lactoferrin is about 3 mg/g of total protein or less, more preferably about 1 mg/g of total protein or less and most preferably less than 1 mg/g total protein, such as 0.3 mg/g or less.

In one preferred embodiment, alpha-lactalbumin (a-Lac) is present in the Ig composition derived from the colostrum of a bovine at no more than about 75 mg/gram of total protein and preferably no more than about 20 mg per gram of total protein present in the composition wherein the total protein content of the composition is measured by bicinchonic acid (BCA) assay and the level of a-Lac is measured by ELISA. More preferably the level of a-Lac is about 3 mg/g (w/w) of total protein or less, more preferably about 1 mg/g or less of total protein and most preferably less than 1 mg/g total protein.

In one preferred embodiment, beta-lactoglobulin (b-Lac) is present in the Ig composition at no more than about 20 mg/g and preferably no more than about 10 mg per gram of total protein present in the composition wherein the total protein content of the composition is measured by bicinchonic acid (BCA) assay and the level of b-Lac is measured by ELISA. More preferably the level of b-Lac is about 5 mg/g or less of total protein, and more preferably about 3 mg or less of total protein, more preferably about 1 mg/g total protein or less and most preferably less than 1 mg/g total protein.

In one embodiment, lactoperoxidase (LPO) is present in the Ig composition at no more than about 10 mg per gram of total protein present in the composition wherein the total protein content of the composition is measured by bicinchonic acid (BCA) assay and the level of LPO is measured by ELISA. More preferably the level of LPO is about 2 mg/g (w/w) of total protein or less, more preferably about 1 mg/g total protein, more preferably about 0.2 mg/g total protein or less and most preferably less than 0.2 mg/g total protein.

In one embodiment, insulin-like growth factor-1 (IFG-1) is present in the Ig composition derived from the colostrum of a bovine at no more than about 10 mg per gram of total protein present in the composition wherein the total protein content of the composition is measured by bicinchonic acid (BCA) assay and the level of IGF-1 is measured by ELISA. More preferably the level of IFG-1 is about 1 mg/g of total protein or less, more preferably about 0.1 mg/g total protein or less and most preferably less than 0.1 mg/g total protein.

In one embodiment, the invention provides processes for preparing a composition comprising isolated and purified immunoglobulin derived from the colostrum of a bovine that has been immunized with all or a portion of a target antigen, wherein the composition is at least 90% immunoglobulin as determined by reducing SDS-PAGE/densitometry and is substantially depleted of non-immunoglobulin factors including but not limited to lactoferrin (LF), alpha-lactalbumin (a-Lac), beta-lactoglobulin (b-Lac), lactoperoxidase (LPO) and insulin-like growth factor-1 (IGF-1) and wherein the composition binds a target antigen in standard antibody binding assays, wherein the preparation of the composition comprises the steps of: providing whey derived from the colostrum of a bovine immunized with a target antigen that has been processed to deplete the fat and casein by standard procedures as is known in the art; adjusting the pH of the processed whey to a pH of 6.6 to 7.0; filtering the whey through an anion exchange column connected in series with a cation exchange column wherein the whey sequentially flows through both columns connected in series without addition of materials that change the salt concentration or pH; collecting the flow through after it sequentially passes through both columns connected in series without addition of materials that change the salt concentration or pH before collection occurs; and concentrating the flow through by ultrafiltration. The process may further comprise affinity purification of the flow through material that has been concentrated by ultrafiltration using, for example, an affinity matrix coupled to the target antigen such as hTNF. The process may further comprise lyophilizing or spray-drying the concentrated flow through product using standard techniques. The process may further comprise testing the concentrated flow through product to determine that the impurities are at desired levels prior to spray drying or lyophilizing by standard means including the assays described in the Examples.

In one embodiment, the specific activity of anti-hTNF polyclonal antibodies present in the whey is increased by about 2 fold after the flow through has been concentrated by the ultrafiltration step. In one embodiment, the neutralizing activity of hTNF cytotoxicity as measured in a standard in vitro L929 assay of affinity purified material is increased by at least 10 fold, preferably at least 100 fold and preferably about 300 fold or more as compared to the hTNF neutralizing activity of the concentrated flow through after ultrafiltration, where neutralizing activity is expressed based on activity per mg of protein.

In one embodiment, the anion exchange column is a strong anion exchanger and the cation exchange column is a strong cationic exchanger column. Strong cation exchangers suitable for use in this invention include but are not limited to Capto S (GE Healthcare Bio-Sciences, Piscataway, N.J.), ToyoPearl GigaCap S-650 M (Tosoh Bioscience, Tokyo, Japan), S Sepharose XL (GE Healthcare Bio-Sciences, Piscataway, N.J.), MacroPrep High S (Bio-Rad Laboratories, Hercules, Calif.), TSK Gel BioAssist S (Tosoh Bioscience, Tokyo, Japan), POROS XS (Life Technologies/Applied Biosystems, Carlsbad, Calif.). Strong anion exchangers suitable for use in this invention include but are not limited to Capto-Q (GE Healthcare Bio-Sciences, Piscataway, N.J.), ToyoPearl GigaCap Q-650 M (Tosoh Bioscience, Tokyo, Japan), Q Sepharose XL (GE Healthcare Bio-Sciences, Piscataway, N.J.), Macro-Prep High Q (Bio-Rad Laboratories, Hercules, Calif.), TSK gel BioAssist Q (Bio-Rad Laboratories, Hercules, Calif.), TSK gel QAE-25SW (Bio-Rad Laboratories, Hercules, Calif.), POROS HQ (Life Technologies/Applied Biosystems, Carlsbad, Calif.).

Weak cation and anion exchangers would also be suitable for use in this invention. Weak cation exchangers suitable for use in this invention include but are not limited to Macro-Prep CM (Bio-Rad Laboratories, Hercules, Calif.), CM Ceramic Hyper D (Pall Corporation, Port Washington, N.Y.), CM Sepharose FF (GE Healthcare Bio-Sciences, Piscataway, N.J.). Weak anion exchangers suitable for use in this invention include but are not limited to TSK-gel DEAE 5PW (Tosoh Bioscience, Tokyo, Japan), TSK-gel DEAE 5NPR (Tosoh BioScience, Tokyo, Japan), Capto-DEAE (GE Healthcare Bio-Sciences, Piscataway, N.J.), DEAE Ceramic Hyper-D (Pall Corporation, Port Washington, N.Y.), Mustang S (Pall Corporation, Port Washington, N.Y.), POROS D (Life Technologies/Applied Biosystems, Carlsbad, Calif.).

In one embodiment the conductivity of the whey solution entering the column is about 4+/−1 milliSiemens/cm. In one embodiment, the conductivity of the flow through of both columns is about 4+/−1 milliSiemens/cm. In one embodiment, the pH of the whey solution entering the column is the same as the pH of the flow through of both columns.

This method is particularly useful in the preparation of large scale amounts of a purified and isolated Ig composition of the invention substantially depleted of non-Ig factors as described above. Depletion of non-immunoglobulin factors from an Ig composition comprising polyclonal antibodies using ion exchange chromatography has been challenging in the past due to the range of pIs of the various antibody clones within the polyclonal composition. Previous methods have required using multiple columns with varying conditions and elution steps to separate the immunoglobulin from the non-immunoglobulin factors having pIs above or below those of the polyclonal antibody species. The use of sequential flow through anionic and cationic ion exchange columns connected in series provides for large scale purification of polyclonal antibodies while simultaneously substantially depleting non-Ig factors from the final composition. This method allows for purification and isolation of Ig compositions without the need for multiple columns, separate elutions and multiple changes in process conditions such as pH, salt and temperature. As used herein large scale purification means at least 30 L liters of starting material (colostrum).

In one preferred embodiment, the invention provides pharmaceutical formulations comprising an optional, pharmaceutically acceptable excipient as is described in detail herein and a composition consisting essentially of isolated and purified immunoglobulin derived from the colostrum of a bovine that has been immunized with all or a portion of a target antigen, wherein the composition is at least 90% immunoglobulin as determined by reducing SDS-PAGE/densitometry and contains less than about 10 mg of lactoferrin per gram of total protein present in the composition and wherein the total protein content of the composition is measured by bicinchonic acid (BCA) assay and the level of lactoferrin is measured by ELISA, wherein the composition binds or modulates the target antigen in an assay. The pharmaceutical compositions of the invention may be depleted of additional non-immunoglobulin factors as described above including but not limited to depletion of alpha-lactalbumin (a-Lac), beta lactoglobulin (b-Lac), lactoperoxidase (LPO) and insulin-like growth factor-1 (IGF-1) to the levels as described herein.

The compositions of the invention comprising polyclonal antibodies specific for hTNF derived from bovine animals that have optionally been depleted of non-Ig factors have several therapeutic and diagnostic applications and uses in competitive binding assays.

In one embodiment the compositions comprising polyclonal anti-hTNF antibodies of the invention are useful as pharmaceutical compositions for the treatment of various diseases in which TNF production contributes to the disease pathology.

In one embodiment the disease is an inflammatory disease such as inflammatory bowel disease or other inflammatory diseases of the digestive tract. In one embodiment, the compositions comprising polyclonal anti-hTNF antibodies of the invention are formulated as pharmaceutical compositions for use in treating diseases of the digestive tract such as inflammatory bowel disease including Crohn's disease and ulcerative colitis, mucositis and damage to the digestive tract resulting from exposure to therapeutic or non-therapeutic radiation.

In one embodiment, compositions comprising polyclonal anti-hTNF antibodies of the invention are suitable for use in the treatment of oral or intestinal mucositis. The mucositis may, for example, be caused by radiation therapy, chemotherapy or any combination thereof. In one embodiment, the mucositis may be caused by exposure to high doses of radiation, including total body irradiation, outside of the context of radiation therapy. In one embodiment, non-Ig factor-depleted anti-TNF polyclonal antibody compositions of the invention are suitable for use in the treatment of recurrent aphthous stomatitis. In another embodiment, non-Ig factor-depleted anti-TNF polyclonal antibody compositions of the invention are suitable for use in the treatment of eosinophilic esophagitis, eosinophilic gastritis, and other conditions involving hypereosinophilic activity in a part of the gastrointestinal tract. Compositions of the invention may be administered topically, for example to the oral cavity to treat oral mucositis and aphthous stomatitis, or orally or rectally to the digestive tract, for example to treat intestinal mucositis.

For some diseases of the digestive tract, treatments are already available. For example, both small molecule and biological therapies are available for the treatment of Crohn's disease and ulcerative colitis, the two forms of inflammatory bowel disease. Most antibody therapies in current use are monoclonal antibodies designed to be delivered systemically and are administered to patients by injection. Injected antibodies have been shown to be useful in the treatment of inflammatory bowel disease, and may also be useful in the treatment of other diseases of the digestive tract. However, administration of antibodies systemically may affect physiological processes throughout the body, rather than just within the digestive tract, and this may be disadvantageous for some diseases.

For instance, anti-TNF antibodies used for the treatment of inflammatory bowel disease are associated with serious side effects. The serum levels of anti-TNF antibody, for example, associated with clinical benefit are concentrations above 0.5 ug/ml (Nestorov, 2005, J Rheumatol Suppl, 74, 13-8; Tracey et al., 2008, Pharmacol Ther, 117, 244-79). While the serum levels of anti-TNF antibody associated with adverse events is not precisely known, it is thought to be greater than the levels needed for clinical benefit (Nestorov, 2005, J Rheumatol Suppl, 74, 13-8). Thus, as compared to existing parenteral TNF antagonists the therapeutic compositions and compositions and methods of the present invention are associated with reduced systemic immunosuppression, reduced systemic distribution, reduced immunogenicity, reduced tachyphylaxis (whether caused by the induction of neutralizing antibodies or by another mechanism) and reduced immediate side effects (e.g. infusion reactions).

When delivered topically to the digestive tract such as by oral or rectal delivery, the bovine-derived polyclonal anti-hTNF compositions of the present invention minimize the activity of the therapeutic antibody outside of the digestive tract and further minimize the induction of a neutralizing immune response against the therapeutic antibody. The antibodies may cross the mucosal barrier of the digestive tract to enter the submucosal space to interact with their targets, but do not substantially enter the systemic circulation.

In one aspect, the invention provides methods of treating a patient using the polyclonal antibody compositions of the invention. The term “patient” as used herein refers to an animal. Preferably the animal is a mammal. More preferably the mammal is a human. A “patient” also refers to, for example, dogs, cats, horses, cows, pigs, guinea pigs, fish, birds and the like.

The terms “treatment”, “treat” and “treating” encompasses alleviation, cure or prevention of at least one symptom or other aspect of a disorder, disease, illness or other condition (collectively referred to herein as a “condition”), or reduction of severity of the condition, and the like. A composition of the invention need not affect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. In one embodiment, an indication that a therapeutically effective amount of a composition that has been administered to the patient who experiences a sustained improvement over baseline of an indicator that reflects the severity of the particular disorder constitutes a treatment for that disorder.

The pharmaceutical compositions of the present invention comprise a therapeutically effective amount of compositions comprising bovine derived anti-hTNF polyclonal antibodies of the present invention formulated together with one or more pharmaceutically acceptable carriers or excipients. By a “therapeutically effective amount” of an antibody of the invention is meant an amount of the composition which confers a therapeutic effect on the treated subject, at a reasonable benefit/risk ratio applicable to any medical treatment. The therapeutic effect is sufficient to “treat” the patient as that term is used herein.

For disorders of the oral cavity, the bovine derived polyclonal anti-hTNF compositions of the present invention of the invention can be delivered in a mouthwash, rinse, paste, gel, or other suitable formulation. Antibodies of the invention can be delivered using formulations designed to increase the contact between the active antibody and the mucosal surface, such as buccal patches, buccal tape, mucoadhesive films, sublingual tablets, lozenges, wafers, chewable tablets, quick or fast dissolving tablets, effervescent tablets, or a buccal or sublingual solid. For disorders of the digestive tract, antibody can be delivered by oral ingestion in the form of a capsule, tablet, liquid formulation or similar form designed to introduce drug to the digestive tract. Alternatively, antibody may be administered by suppository or enema for delivery to the lower digestive tract. Such formulations are well known to those skilled in the art.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound. In one embodiment, compositions for rectal administration are in the form of an enema.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

It may be desirable under some conditions to provide additional levels of protection against gastric degradation of orally or rectally delivered compositions of the invention. If this is desired, there are many options for enteric coating. In one embodiment, enteric coatings take advantage of the post-gastric change in pH to dissolve a film coating and release the active ingredient. Coatings and formulations have been developed to deliver protein therapeutics to the small intestine and these approaches could be adapted for the delivery of an antibody of the invention. For example, an enteric-coated form of insulin has been developed for oral delivery {Toorisaka et al., 2005, J Control Release, 107, 91-6}.

In addition, the solid dosage forms of tablets, dragees, capsules, pills, and granules can be prepared with other coatings and shells well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Effective doses will vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the timing of delivery of the compound relative to food intake; the duration of the treatment; drugs used in combination or contemporaneously with the specific compound employed; and like factors well known in the medical arts.

Particular embodiments of the present invention involve administering a pharmaceutical composition comprising an antibody of the invention at a dosage of from about 1 mg per day to about 1 g/day, more preferably from about 10 mg/day to about 500 mg/day, and most preferably from about 20 mg/day to about 100 mg/day, to a subject. In one embodiment, a polyclonal antibody preparation is administered at a dosage of antibody from about 100 mg to about 50 g/day, more preferably from about 500 mg/day to about 10 g/day, and most preferably from about 1 g/day to about 5 g/day, to a subject, wherein the polyclonal antibody preparation has not been enriched for antibodies specific for the target antigen.

Treatment regimens include administering an antibody composition of the invention one time per day, two times per day, or three or more times per day, to treat a medical disorder disclosed herein. In one embodiment, an antibody composition of the invention is administered four times per day, 6 times per day or 8 times per day to treat a medical disorder disclosed herein. In one embodiment, an antibody composition of the invention is administered one time per week, two times per week, or three or more times per week, to treat a medical disorder disclosed herein.

The methods and compositions of the invention include the use of an antibody of the invention in combination with one or more additional therapeutic agents useful in treating the condition with which the patient is afflicted. Examples of such additional agents include both proteinaceous and non-proteinaceous drugs. Non-limiting examples of such additional therapeutic agents for, e.g., inflammatory bowel disease, with which an antibody of the invention can be combined include the following: oral steriods, IFN-β, budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; CDP-571/BAY-10-3356 (humanized anti-TNF antibody; Celltech/Bayer); 75 kdTNFR-IgG (75 kD TNF receptor-IgG fusion protein; Immunex; see e.g., Arthritis & Rheumatism (1994) Vol. 37, 5295; J. Invest. Med. (1996) Vol. 44, 235A); 55 kdTNFR-IgG (55 kD TNF receptor-IgG fusion protein; Hoffmann-LaRoche); interleukin-10 (SCH 52000; Schering Plough); IL-4; IL-10 and/or IL-4 agonists (e.g., agonist antibodies); interleukin-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of Platelet Activating Factor (PAF); ciprofloxacin; and lignocaine.

Many potential co-therapeutic agents suitable for treating TNF modulated disease (e.g. IBD) are commercially available or are currently in clinical development and include the following: 5-ASA (generic); MMX Mesalazine (Cosmo); MMX Budesonide (Cosmo); MMX LMW Heparin (Cosmo); ER Mesalazine (Salix); Azathioprine (generic); 6-mercaptopurine; Infliximab (Centocor, J&J); Adalimumab (Abbott); Certolizumab pegol (UCB); Atrosab (BalioPharma); Natalizumab (Elan); Golimumab (Centocor J&J); Dersalazine (Palau); HMPL-004 (Hutchinson Medi Pharma); Ozoralizumab (Ablynx); TNF-a Kinoid (Neovacs); Apilimod (Synta); Ustekinumab (Centocor J&J); Briakinumab (Abbott); SCH-900222 (Schering Plough); FM202 and FM303( ) MP-196; Basiliximab (Cerimon); Daclizumab (Roche); Fontolizumab (PDL); C326 (Avidia); Sirukumab (Centocor J&J); Olokizumab (UCB); Sarilumab (Centocor J&J); BMS-945429 (Alder); Tocilizumab (Chugai); Anrukinzumab (Wyeth); QAX567 (Novartis); GSK1070806 (GSK); PF-05230900 (Pfizer) Vidofludimus (4SC); Tofactinib (Pfizer); AG014 (Actogenix); IL-27 ActoBiotic (Actogenix); Visilizumab (PDL); Rituximab (Genentech); Abatacept (BMS); Filgrastim (Amgen); Sargramostim (Immunex, Amgen); Vidolizumab (Millennium); Etrolizumab (Genentech); AJM-300 (Ajinimoto); ASP-2002 (Mitsubishi); Alicaforsen (Isis); PF-547659 (Pfizer); CCX282 (GSK1605786); CCX507 (ChemoCentryx); CNDO-201 (Coronado); Remestemcel-L (Osiris); PDA-001 (Celgene); OvaSave (TxCell); Secukinumab; MDX-1100 (Medarex); Tetomilast (Otsuka); LT-02 (Lipid Therapeutics); VT-301 (ViThera).

When multiple therapeutics are co-administered, dosages may be adjusted accordingly, as is recognized in the pertinent art. “Co-administration” and “combination therapy” are not limited to simultaneous administration, but also include treatment regimens in which an antibody of the invention is administered at least once during a course of treatment that involves administering at least one other therapeutic agent to the patient.

The following examples are provided for the purpose of illustrating specific embodiments or features of the invention and are not intended to limit its scope.

### EXAMPLES

### Example 1

### Bovine Colostral Anti-TNF Polyclonal Antibody Composition and Process

Immune colostrum is produced at an audited, qualified animal facility. Pregnant Holstein dairy cows are sourced from commercial Grade A dairies in the US which are regulated under the FDA Pasteurized Milk Ordinance (PMO). The PMO specifies housing requirements, building and equipment standards, use of acceptable cleaning and pesticide materials, milking procedures, sanitation requirements, etc.

Animals are quarantined for a minimum of two weeks prior to the start of immunizations and dried off if necessary. Qualified cows are housed and maintained separately from other animals and observed daily. Feed sources are controlled to prevent the introduction of unapproved animal source protein. Source dairy herds are tested or certified by the state to be free of brucellosis and TB. Cows receive (killed or inactivated) routine immunizations for, or are screened for:

| | |
|---|---|
| Bovine leukemia virus | E. coli |
| Bovine viral diarrhea virus | Rotavirus |
| Parainfluenza virus (PI₃) | Vibriosis |
| Infectious bovine rhinotracheitis | Leptospirosis |
| Bovine respiratory syncytial virus | Clostridial diseases |
| Mycobacterium paratuberculosis | Coxiella burnetti. |
| Bovine rabies | |

Qualified cows are immunized with three (3) doses of rhTNF using commercial veterinary adjuvants that have been USDA approved for use in dairy cows. Final prepared vaccines are administered under the direct supervision of a veterinarian according to established SOPs at intervals of two to three weeks. Serum samples are collected at the time of each injection and at calving.

Immunized cows are milked individually. Animals must be in apparent good health at calving with no evidence of clinical mastitis. The cow's udder is prepared for milking using standard dairy cleaning practices and materials approved for use under the FDA Pasteurized Milk Ordinance. Colostrum is collected twice daily for three (3) days after parturition. A sample of each individual colostral milking is collected for analysis and both samples and bulk colostrum are immediately frozen at −20° C. All incoming raw colostrum is qualified before use.

Colostrum is thawed and the fat component is reduced by continuous flow centrifugation at a flow rate of 1200 to 3600 lb/hr and a temperature of 24° to 43.5° C. The skim is diluted with 1.5 volumes of reverse osmosis (RO) water, the pH measured and recorded, and then adjusted to 4.6±0.1 with acid. The acidified skimmed colostrum is allowed to remain quiescent for 25-45 minutes at a temperature of 21° to 35° C. The casein precipitated by the acidification step is removed by decanting centrifugation. Clarified supernatant and casein sludge are collected separately, measured and recorded, and the casein fraction discarded.

Immunoglobulins from the clarified supernatant are isolated by Protein G chromatography in a closed system. Protein G resin (e.g. Sepharose 4 Fast Flow gel, Pharmacia Biotech AB, Uppsala, Sweden) is packed into a column and equilibrated with binding buffer as recommended by the manufacturer. To ensure proper ionic strength and pH are maintained for optimal binding, the clarified supernatant is dialyzed against binding buffer and then applied to the bed volume at a ratio of total protein to bed volume of 20 mg/ml. Flow rate is 0.8 ml/min. The column is washed with 10 bed volumes of the binding buffer. Bound bovine IgG is eluted with 10 bed volumes of 0.1 M glycine-HCl buffer (pH 2.7). To neutralize the eluted fractions, 100 μl/ml of 1M Tris-HCl (pH 9.0) is added to the collection tubes prior to the elution. The purification profile is monitored at 280 nm and target fractions collected, pooled and dialyzed against PBS at 4° C. The collected product eluate is concentrated by ultrafiltration.

### Example 2

### Bovine Colostral Anti-TNF Antibody: Comparison of Purified Antibody with Immune Colostral Whey in a Mouse Model of Inflammatory Bowel Disease

Immune colostrum was produced at Southwest Biolabs, a USDA-registered research facility in Las Cruces, N. Mex. Six Holstein cows were purchased during their last trimester of pregnancy, transported to the facility, and acclimatized for one week prior to immunization. The animals received 3 subcutaneous injections of antigen with one of two adjuvants, spaced 2-3 weeks apart, with the last injection given three weeks prior to the calculated date of parturition. Colostrum was collected from all animals for the first 8 milkings (first four days after calving). One animal calved prematurely, before full udder development had occurred, resulting in low levels of immunoglobulin in the colostrum, and colostrum from this animal was discarded.

A pool was prepared from colostrum collected on days 1-4 post-parturition and whey was prepared using standard methods (Su and Chiang, 2003). Colostrum was diluted 1:3 with distilled water, acidified to pH 4.6 with glacial acetic acid to precipitate casein, and centrifuged. The supernatant was removed and the pH was adjusted to 7.4 to generate immune whey.

The immunoglobulin fraction was purified using thiophilic adsorbent. Thiophilic adsorbent (T-gel) was purchased from Pierce (Thermo Scientific). A chromatography column was packed with 50 ml of resin and equilibrated with 150 ml binding buffer (0.5 M sodium sulfate, 20 mM sodium phosphate, pH 8.0). Immune whey was thawed in a water bath and solid sodium sulfate added to bring the final concentration to 0.5 M. The solution was spun at 3700 rpm for 15 minutes to remove particulate matter, diluted 1:1 with binding buffer and loaded onto the T-gel column at room temperature. The column was washed with 5 column volumes (150 ml) of binding buffer. Immunoglobulin was eluted with low salt (50 mM sodium phosphate pH 8.0) and column fractions containing protein were eluted and pooled. The eluted material was concentrated on an Amicon stirred cell with a YM filter with a 100,000 molecular weight cutoff and filter sterilized.

Control immunoglobulin was purified in parallel. Both immunoglobulin containing anti-TNF activity (interchangeably referred to herein as “murine AVX-470” or “AVX-470m”) and control colostral immunoglobulin were assayed for their ability to both bind to and neutralize murine TNF. Immune immunoglobulin bound to TNF in a specific ELISA, while no binding was seen with control immunoglobulin.

The ability of the bovine antibody to neutralize TNF-induced cytotoxicity was determined using a standard cell-based TNF-assay using murine L929 cells. Varying concentrations of antibody were preincubated with murine TNF for 2 hr at 37° C. in a 96 well microtiter plate. The antibody-antigen mixture was added to confluent cultures of L929 cells along with 1 ug/ml actinomycin D and incubated at 37° C. for 24 hr. Cell viability was assessed using the WST assay. Anti-TNF antibody neutralized TNF in this cell based assay, while the control antibody had no effect.

The purified AVX-470m and control immunoglobulin, along with whey from cows immunized with murine TNF (AVX-470m-whey) and control whey, were evaluated in the murine TNBS-induced colitis model. The study was performed at Biomodels, LLC. Male C57Bl/6 mice with average starting body weight of 21.0 g were obtained from Charles River Laboratories (Wilmington, Mass.). Mice were acclimatized for 5 days prior to study commencement. Colitis was induced by the intrarectal administration of 4 mg of TNBS in a 50% ethanol vehicle on day 0.

Colitis was induced by intrarectal administration of 100 μL of TNBS (4 mg) in 50% ethanol under isoflurane anesthesia on day 0. Eight additional animals served as untreated controls and were dosed intrarectally with 100 μL of 50% ethanol. Animals were dosed with test article or vehicle twice a day (b.i.d.) at 0.1 mL per dose, from day −1 to day 3 via oral gavage (p.o.). On day 5 colitis severity was assessed in all animals using video endoscopy. Endoscopy was performed in a blinded fashion using a small animal endoscope (Karl Storz Endoskope, Germany). To evaluate colitis severity, animals were anesthetized with isoflurane and subjected to video endoscopy of the lower colon. Colitis was scored visually on a scale that ranges from 0 for normal, to 4 for severe ulceration. In descriptive terms, this scale is defined as follows:

### Endoscopy Colitis Scoring Scale

### Score: Description

### 0: Normal

1: Loss of vascularity
2: Loss of vascularity and friability
3: Friability and erosions
4: Ulcerations and bleeding
Statistical differences between a test group and the vehicle control were determined using a Student's t-test (SigmaPlot 11.2, Systat Software, Inc.). The endoscopy scores are shown below in Table 5.

TABLE 5

| | | | Difference from |
|---|---|---|---|
| | | | TNBS-vehicle |
| | Ave | St Dev | control |
| EtOH control | 0.25 | 0.46 | p < 0.001 |
| TNBS-vehicle | 2.17 | 0.58 | NA |
| 5 mg AVX-470m | 1.50 | 0.76 | p = 0.038 |
| 1.5 mg AVX-470m | 1.75 | 0.71 | NS |
| 0.5 mg AVX-470m | 1.75 | 1.28 | NS |
| 1.5 mg Control Ig | 2.50 | 0.76 | NS |
| AVX-470m-whey | 1.63 | 0.52 | p = 0.046 |
| Control whey | 2.00 | 0.53 | NS |

Colitis scores were significantly elevated in the groups treated with TNBS compared to the ethanol-treated control group. Groups receiving oral treatment with 5 mg AVX-470m or AVX-470m-whey both displayed significantly reduced colitis severity scores on day 5. No other significant differences in colitis severity were observed.

Surprisingly, these data demonstrate that activity is seen both with AVX-470m-whey and with purified AVX-470m; no diminution of activity is seen when the immunoglobulin is purified away from the other whey components.

### Example 3

### Production of Immune Colostrum

Immune colostrum was produced at an audited, qualified animal facility. Pregnant Holstein dairy cows were sourced from commercial dairy farms regulated under the US FDA Grade A Pasteurized Milk Ordinance (PMO). Animals were quarantined and dried off. Source dairy herds were tested or certified by the state to be free of brucellosis and TB. Cows received (killed or inactivated) routine immunizations for, or were screened for:

| | |
|---|---|
| Bovine leukemia virus | E. coli |
| Bovine viral diarrhea virus | Rotavirus |
| Parainfluenza virus (PI₃) | Vibriosis |
| Infectious bovine rhinotracheitis | Leptospirosis |
| Bovine respiratory syncytial virus | Clostridial diseases |
| Mycobacterium paratuberculosis | Coxiella burnetti. |
| Bovine rabies | |

Qualified cows were immunized with three (3) doses of rhTNF using Quil A adjuvant at two to three week intervals with the last injection given three weeks prior to the calculated date of parturition. Colostrum was collected from all animals for the first 8 milkings (first four days after calving). A sample of each individual colostral milking was collected for analysis and both samples and bulk colostrum were immediately frozen at −20° C. All cows produced specific antibody as judged by specific binding to recombinant human TNF by ELISA and neutralization of recombinant human TNF in the L929 cell assay.

### Example 4

### Purification of Immunoglobulin from Bovine Colostrum by Ammonium Sulfate Precipitation

Colostrum samples from cows immunized with recombinant murine TNF were thawed and combined to generate a pool of 750 mL of colostrum. To remove fat, the colostrum was centrifuged at 2954×g for 20 minutes at room temperature. After fat removal, the colostrum was diluted in water (1 part colostrum; 2 parts water), and the pH was adjusted to 4.6 using acetic acid, then stirred for 20 minutes. The suspension was centrifuged at 3488×g for 30 minutes at room temperature and the casein pellet was removed from the whey. The pH of the whey was adjusted to pH 7.4 using 10N NaOH. A 50% saturated ammonium sulfate solution (313 g/L of ammonium sulfate) was slowly added to the whey and stirred for 1.5 hours at 4° C. The suspension was centrifuged at 3488×g for 30 minutes at 4° C. The supernatant was slowly decanted. The immunoglobulin pellet was resuspended in phosphate buffered saline (PBS, pH 7.2) to dissolve the pellet. The samples were dialyzed against 8 changes of 2 L of PBS (pH 7.2) at 4° C. for 36 hours. Bovine immunoglobulin was concentrated by adding polyvinylpyrrolidone powder (PVP-40, SIGMA-Aldrich, St Louis, Mo.) on top of the tubes at 4° C. The concentrated immunoglobulin solution was removed from the dialysis tubes.

### Example 5

### Removal of an Impurity from Colostrum on a HiTrap Capto S Column

Frozen colostrum (1.89 L) was thawed in a water bath at 45° C. Following an acidification step with acetic acid to precipitate casein, the colostrum preparation was held overnight at 4° C. The acidified material was warmed to 43° C. and centrifuged at 2,730 RCF. The supernatant was retained and neutralized to pH 6.4 with sodium hydroxide. The neutralized preparation was diluted by adding an equal volume of reverse osmosis water to produce 2.8 L of defatted, casein-reduced colostrum or colostral whey. Aliquots of the whey preparation were tested to evaluate the effectiveness of various chromatography columns.

In this example, an aliquot (30 mL) of the whey was applied to an anion exchange column (5 mL HiTrap Capto Q packed column, purchased from GE Healthcare Bio-Sciences, Piscataway, N.J.) or a cation exchange column (5 mL HiTrap Capto S, also from GE Healthcare Bio-Sciences). Each column was eluted with 1 M NaCl, and the flow through and eluate were analyzed by SDS-PAGE under reducing conditions. Marker lanes were loaded with Dual Color Molecular Weight Marker (Bio-Rad Laboratories, Hercules, Calif.). The gel was stained with Coomassie Blue R-250 to visualize proteins. The two bands at 50 kDa and 25 kDa indicated the heavy and light chains, respectively, of immunoglobulin. The data from the gel showed that during the preparation of the whey, there was no significant yield loss of immunoglobulin as judged by this method. Immunoglobulin is visualized in the flow through of both the Capto-Q and Capto-S columns. Under the conditions used for this colostral whey preparation, the Capto-Q matrix did not significantly bind any abundant protein in the whey preparation, although it can have an important role in removal of less abundant protein impurities, as seen in further examples. By contrast, Capto-S was noted to bind and thus concentrate a protein from colostral whey with a reduced molecular weight of approximately 75 kDa.

### Example 6

### Preparation of Polyclonal Antibody Composition by Depleted of Non-Immunoglobulin Factors by Mercapto-Ethyl-Pyridine (MEP) Chromatography

MEP matrix (Pall Corporation, Port Washington, N.Y.), useful for the purification of immunoglobulins, was tested for its ability to purify the polyclonal antibody preparation from whey. In this example, a 25 mg sample from the Capto-S flow through was adjusted to a final concentration of 0.15 M NaCl and filtered with an 0.22 μm filter (Millipax, Millipore Corporation, Billerica, Mass.). The sample was then applied to a 1 mL column of MEP matrix at a flow rate of 2 mL/min. Absorbance at 280 nm was monitored, and the column was washed until absorbance units reached baseline levels. Protein that bound to the column was eluted with a gradient of citric acid to decrease the pH. The immunoglobulin fraction eluted at approximately pH 5.0. The gel was prepared as follows: Lane 1 is the BioRad Precision Dual Color marker, Lane 2, the Capto-S flow through (MEP column load), Lanes 3-12 fraction 36-43 inclusive fractions from the elution peak. A densitometry scan quantitated using ImageJ software (NIH, http://rsb.info.nih.gov/ij/index.html) revealed that the heavy and light chains accounted for approximately 95% of the total protein.

These data suggest that MEP may be an effective resin for removing impurities. However, later examples will demonstrate that MEP is not the preferred method.

### Example 7

### Investigation of the Composition of the MEP-Purified Whey Protein Antibody Preparation by Analytical Size Exclusion Chromatography

Size exclusion chromatography is a useful technique for assessing the composition of purified protein preparations. Protein complexes or proteins with higher native molecular weight elute earlier than proteins with lower native molecular weight. Pooled MEP eluate from the chromatography of whey protein (0.5 mg in a total volume of 0.5 mL) was subjected to analytical size exclusion chromatography analysis on a high resolution TRICORN®S200 Column (Superdex 200 10/300 GL, from GE Healthcare Bio Sciences, Piscataway, N.J.) on an ÄKTAEXPLORER™ FPLC system. The column was pre-equilibrated in phosphate buffered saline (0.15M NaCl), which was also the elution buffer. Absorbance was monitored at 280 nm. Area under the peaks was measured using the Unicorn software package. Under these conditions, the immunoglobulins were expected to maintain native conformation. The data showed a primary peak with elution volume of 13.5 mL was calculated to represent a retention time of approximately 149 kDa for a globular protein, very close to the theoretical molecular weight 150 kDa molecular weight for an immunoglobulin. The data in this example are consistent with the SDS-PAGE analysis and show that the MEP matrix bound and purified the polyclonal antibody composition.

### Example 8

### Investigation of Scale Up to Larger Scale Process Using 15 L Colostrum and a 2 L MEP Column

In this example, parameters were investigated in order to scale up the MEP column process. Defatted whey was prepared at pilot scale: first, defatted colostrum (15 L) was prepared by continuous flow centrifugation, followed by acidification to pH 4.6 with 10% lactic acid. After an overnight hold, the casein was removed by centrifugation and the supernatant was retained and neutralized to pH 6.4 with 0.5 M NaOH. The whey was then filtered through a pilot scale filter train, a depth filter (CUNO Zeta Plus filter Cartridge) followed by a 0.2 μm filter, and loaded onto a 2 L column of MEP resin packed into an INdEX column preequilibrated with 20 mM citrate-phosphate buffer, pH 6.8. The column was extensively washed with approximately 10 L of the same buffer, and then eluted with 20 mM citrate-phosphate, pH 2.8. The eluted sample was neutralized with 1 M Tris. The eluate was then diafiltered versus 5 volumes of reverse osmosis water to exchange the buffer, and then concentrated by ultrafiltration using a Pilot Scale Tangential Flow Filtration Apparatus (Pall Corporation, Port Washington, N.Y.). Viscosity was not observed to be a problem.

The results from the reducing SDS PAGE analysis largely recapitulated the results seen at the bench scale. Two major impurities were noted to be present in addition to immunoglobulin heavy and light chain.

### Example 9

### Spray Drying Example

Eluate from MEP chromatographic separation of bovine immunoglobulin was concentrated by ultrafiltration/diafiltration to approximately 80 mg/ml protein to create the feedstream for bench scale spray drying experiments. All spray drying development work was conducted by Pharma Spray Drying, Inc. Bedford Hills, N.Y., using a Buchi B-290 bench top lab spray dryer.

The purpose of these initial experiments was to identify spray drying conditions that would form a collectable powder within the cyclone with minimum sticking and product hold up. No excipients were added to the concentrated colostral immunoglobulins prior to spray drying. The parameters for each test are shown in Table 6.

TABLE 6
Buchi B-290 Test Work

| | | Out- | Atm. | | | | |
|---|---|---|---|---|---|---|---|
| | Inlet | let | Air | | | | |
| | Temp. | Temp. | Pres- | | Atm. | Pump | |
| Test | Deg. | Deg. | sure | Fan | Air | set- | |
| # | C. | C. | (bar) | speed | Rate | ting | Results |
| 1 | 110 | 65 | 6 | 100% | 30% | 25 | 1.46 g collected. |
| | | | | | | rpm | Great collection no |
| | | | | | | | sticking in cyclone |
| 2 | 110 | 75 | 6 | 100% | 30% | 10 | 1.25 g collected. |
| | | | | | | rpm | Great collection no |
| | | | | | | | sticking in cyclone |
| 3 | 100 | 60 | 6 | 100% | 30% | 18 | 3.1 g collected. |
| | | | | | | rpm | Great collection |
| | | | | | | | slight sticking in |
| | | | | | | | cyclone |
| 4 | 100 | 50 | 6 | 100% | 30% | 22 | 4.9 g collected. |
| | | | | | | rpm | Great collection |
| | | | | | | | slight sticking in |
| | | | | | | | cyclone |
| 5 | 120 | 80 | 6 | 100% | 30% | 15 | 2.5 g collected. |
| | | | | | | rpm | Great collection no |
| | | | | | | | sticking in cyclone |
| 6 | 120 | 70 | 6 | 100% | 30% | 25 | 1.9 g collected. |
| | | | | | | rpm | Great collection no |
| | | | | | | | sticking in cyclone |
| 7 | 120 | 60 | 6 | 100% | 30% | 32 | 3.4 g collected. |
| | | | | | | rpm | Slight overspray |
| 8 | 120 | 50 | 6 | 100% | 30% | 47 | 2.8 g collected. |
| | | | | | | rpm | Over spraying in |
| | | | | | | | main |
| 9 | 150 | 90 | 6 | 100% | 30% | 18 | 3.0 g collected. |
| | | | | | | rpm | Great collection no |
| | | | | | | | sticking in cyclone |
| 10 | 150 | 75 | 6 | 100% | 30% | 42 | 2.3 g collected. |
| | | | | | | rpm | Great collection no |
| | | | | | | | sticking in cyclone |
| 11 | 100 | 55 | 6 | 100% | 30% | 28 | 2.5 g collected. |
| | | | | | | rpm | Great collection |
| | | | | | | | slight sticking in |
| | | | | | | | cyclone |

Each of these test powders was hand-filled into gelatin capsules (Size 00, Capsugel, Cambridge, Mass.) to produce prototype oral dosage forms.

### Example 10

### Comparison of MEP Purification and Capto-S Purification Processes

In evaluating the results obtained using the MEP resin, there was concern about the presence of impurities in the eluate, as well as concerns about binding capacity. In addition, in a process for preparation of pharmacologic compositions, scalability, rapid throughput, and avoiding changes in volume are important factors. A process whereby the active pharmaceutical ingredient does not bind to a column resin while undesired contaminants do bind may represent a preferred process. Therefore, the flow-through methods were re-examined.

In this example, early steps are performed as described (Gregory, A. G., U.S. Pat. No. 5,707,678): defatted colostrum was diluted 2× with reverse osmosis water, acidified, neutralized, then processed in the continuous flow centrifuge. After an overnight hold step, diatomaceous earth (USP/NF grade, Sigma Aldrich) was added to 4/g L and the material was stirred for 10 min, neutralized with 10% sodium hydroxide, and filtered through a Cuno Zeta Plus BioCap depth filter (602A05A, 3M Corporation, St. Paul, Minn.) and a 0.2 μm filter (MilliPAK MPGL 02GH2, Millipore Corporation, Billerica, Mass.).

The whey was applied to either an MEP column or Capto-S column. Following chromatography, the appropriate fractions from each arm of the comparison (retained fractions, eluted with a pH gradient for MEP; flow through for Capto-S, adjusted to 100 mM NaCl) were then ultrafiltered to an estimated concentration of 50 g/L using a Pall Pharmaceutical series apparatus (Pall Corporation, Port Washington, N.Y.) and TMP-Flux 50 kD nominal molecular weight cut-off (NMWCO) membranes. The trans-membrane pressure (TMP) was adjusted to maintain a level close to 15 psi. The material was diafiltered versus three to five volumes of reverse-osmosis water, followed by a second ultrafiltration step to bring the protein concentration to 100 g/L. Protein concentration was determined by the bicinchoninc acid method using the BCA™ assay kit, carried out as described by the supplier (Thermo Fisher Scientific, Rockford, Ill.). Samples were run on reducing 4-12% Bis-Tris NOVEX Gels (NUPAGE, Invitrogen) using NUPAGE MOPS SDS Running Buffer. Marker lanes were Novex Sharp prestained protein standards (Invitrogen, Carlsbad, Calif.). The gel was stained with the EZ Blue staining reagent (Sigma Cat G1041). Gels were scanned on a desk top scanner (HP ScanJet Model G3010) and imaging data analyzed by ImageJ software (NIH).

Analysis of the gels indicated that the MEP process and Capto-S-TFF processes produced different profiles, for instance with the MEP process having a preponderance of lactoferrin as a likely contaminant and the Capto-S process having lactoglobulin as a likely contaminant. The identity of contaminants was determined by comparison to standards run on SDS-PAGE, consideration of isoelectric points, and results from mass spectrometry analysis. Subsequently, appropriate optimization and polishing steps can be applied to achieve different preferred embodiments of polyclonal antibody compositions.

### Example 11

### Quantitation of IgM and IgA in Polyclonal Compositions

Commercially available ELISA kits (Cat.#E11-101 and #E11-121, Bethyl Laboratories, Montgomery, Tex.) were used to determine the levels of IgM and IgA, respectively, in different preparations. Anti-bovine IgM or IgA antibodies are precoated on the 96-well strip plates provided. The plates were washed, blocked, and serial dilutions of samples were added, washed, and binding detected with either horseradish-peroxidase conjugated, affinity purified goat anti-bovine IgM or goat anti-bovine IgA and 3,3′,5,5′-tetramethylbenzidine (TMB) as substrate. Material purified by MEP chromatography was compared with the flow through material from Capto-S chromatography. Data in Table 7 are expressed as mg of the isotype per gram of product based on protein concentration using the BCA assay.

TABLE 7

| Sample | IgA (mg/g) | IgM (mg/g) |
|---|---|---|
| Defatted colostrum | 24 | 51 |
| MEP | 108 | 14 |
| Capto-S | 136 | 72 |

IgA levels were increased in both purified preparations, reflecting enrichment of immunoglobulin as impurities (particularly casein) is removed. IgM is slightly enriched in the Capto-S preparation, but is significantly depleted in the MEP preparation. This further demonstrates the superiority of the Capto-S method over MEP. In the Capto-S preparation, 13% of the protein was IgA and 7% was IgM, reflecting retention of all IgA and loss of approximately 50% of the IgM, based on typical levels of these isotypes in colostrum.

### Example 12

### Selective Precipitation to Remove Lactoferrin

Selective precipitation is a technique that can concentrate a protein of interest or remove a contaminating protein. In this experiment, it was found that neutralization of acidified, defatted, decaseinated colostrum with dibasic phosphate selectively precipitated lactoferrin. Defatted colostrum was thawed and heated to 42° C. and diluted with 1.5× volumes of water. The solution was acidified with 5% lactic acid to a final pH of 4.6. Casein was removed by crude filtration followed by continuous flow centrifugation and the acidified material was held overnight at 2-8° C. In the morning, 4 g/L diatomaceous earth was added and the material filtered through a CUNO Zeta Plus Capsule filter. Different neutralization conditions were then compared, varying temperature, rate of neutralization, and use of NaOH or Na(P) dibasic. In all cases, some turbidity was observed and precipitated material was removed by centrifugation and analyzed by reducing SDS PAGE.

In this experiment, a 75 kDa protein of the same relative mobility of lactoferrin (compared to a commercially available standard) was found enriched in the pellet fraction when sodium phosphate dibasic was used to neutralize the pH in preparation of whey from post-casein colostrum compared to sodium hydroxide. The relative enrichment of putative lactoferrin was accompanied by a white precipitate, likely to be calcium phosphate.

Based on this result, a pilot scale run was carried out using sodium dibasic phosphate as a neutralization agent and using the continuous flow centrifuge to remove the precipitated material. However, the calcium phosphate precipitate proved to be extremely difficult to clean from the processing equipment. Therefore, although this method may be useful at bench scale, it is not a preferred method for pilot or production scale.

### Example 13

### Advantage of Sequential Flow Through Strategy—Bench Scale Study

The experiment described here shows bench scale chromatography using resins that reliably scale to pilot and process scales, followed by analysis of the protein profiles using reducing SDS PAGE. Colostral whey was prepared at pilot scale and samples were loaded onto 5 ml columns as indicated below and analyzed by SDS PAGE.

Together with the data in Example 10, this experiment suggests a sequential flow through chromatography process with Capto-S and Capto-Q can result in an improved process when compared with MEP column chromatography. In particular, results with the novel strategy of flow through Capto-Q in series with flow through Capto-S looks particularly promising.

### Example 14

### Serial Capto-S and Capto-Q Chromatography Scaled to 30 L Colostrum and 3 L Columns

Fat was removed from 30 L of colostrum by continuous flow centrifugation in a Westphalia apparatus (SA-1-02-175, GEA Mechanical Equipment US, Inc., Northvale, N.J.), acid precipitation by lactate addition at 42° C. (DL-Lactic Acid, 85% solution, (Fisher Scientific, Waltham, Mass.) and crude filtration. Following the crude filtration, the material was held overnight at 2-8° C. and then neutralized by Tromethamine addition (Trizma Base, Sigma Aldrich, St Louis Mo.). The neutralized whey was clarified by continuous flow centrifugation. Next, in a flocculation step, diatomaceous earth filter agent (Sigma Aldrich, St Louis, Mo.) was added to 4 g/mL prior to the first filter capsule (Gregory, A. G., U.S. Pat. No. 5,707,678), with stirring for 10 min. The clarification filter train consisted of a 20 μm Alpha fibrous polypropylene (Meissner Filtration Products, Camarillo, Calif.)/0.45 μm polypropylene filter CLMFO.45-222 (Meissner Filtration Products, Camarillo, Calif.)/0.2 μm filter (Pall Corporation, Port Washington, N.Y.).

Capto-S resin (3 L bed volume) and Capto-Q resin (3 L bed volume) were packed in two INdEX 140/500 columns (GE Healthcare Bio-Sciences Corp., Piscataway, N.J.), connected in series. Prior to loading the sample, the columns were washed sequentially with 12 L reverse osmosis water, 12 L 0.5 M NaCl, 12 L reverse osmosis water, 12 L 1 M NaCl, 12 L reverse osmosis water, then 60 L 1 M Tris-HCl pH 6.8. The whey (30 L) was pumped onto the column at a flow rate of 0.5 L/min, and the column was washed with 2.5 column volumes of equilibration buffer. Absorbance at 280 nm was monitored using an inline flow cell (PendoTECH, Princeton, N.J.). Collection of flow through was stopped when A280 approached baseline levels. After chromatography, the product was concentrated by ultrafiltraton (50 kDa NMWCO filter), using a Pall Pharmaceutical Series apparatus, Pall Corporation, Port Washington, N.Y.) then diafiltered versus 5 volumes of reverse osmosis water. The product was concentrated to >75 mg/mL by ultrafiltration. Terminal heat treatment was performed at 60° C. for 10 hours.

The SDS PAGE analysis was conducted to gather the results from this 30 L pilot scale column chromatography on Capto-S and Capto-Q, connected in series. Proteins bound to Capto-S and Capto-Q columns were assessed by stripping the column with 1 M NaCl. The gel was prepared as follows: Lane 1, Protein Molecular Weight Markers, Lanes 2-4, increasing loads of IgG L-chain standard used as a control to quantify immunoglobulin content (electrophoresis, >99% pure, from human myeloma plasma, obtained from Sigma Aldrich, St Louis, Mo.) Lane 5, load prior to serial chromatography, Lane 6, Flow through from Capto-S/3 L Capto-Q serial columns, Lane 7, Eluate of Serial Columns (1M NaCl). Gels were stained with Coomassie Brilliant Blue and electronically imaged using a MFC-9120CN scanner (Brother). ImageJ 1.45 s software (National Institutes of Health, Bethesda, Md., imagej.nih.hov/ij/docs) was used create densitometry plots. Peak area was measured by integrating the baseline-subtracted area between the half-peak heights.

Comparison of the densitometry traces of lanes 5 and 6 shows diminution of non-Ig proteins and concentration of Ig proteins, heavy and light chains. In the composition shown in lane 6, 81% of the product is present in immuglobulin heavy and light chains. The high molecular weight band is aggregated Ig heavy chain (see Example 14) and the majority of the material present in the 70-80 kDa section is also product-related (IgM and secretory component—see Example 14). Therefore 95% of the product is immunoglobulin.

The trace of Lane 7 showed that a number non-Ig proteins preferably bind to the resins. Taken together with the traces from Lanes 5 and 6 and other data, it was concluded that serial flow through chromatography is a powerful method for preparation of polyclonal antibody compositions from colostrum. The identities of proteins in the flow-through and eluate were investigated further in the examples below. It will be readily recognized that this process or variations thereof will provide the appropriate yields of polyclonal antibody compositions suitable for oral administration.

### Example 15

### Serial Capto-S and Capto-Q Chromatography Scaled to 80 L Colostrum (Prophetic)

Having exemplified the method for preparing antibody compositions at 30 L scale, it will be recognized by those skilled in the art that the procedure can be scaled up to 80 L without extensive experimentation. Preparation of antibody compositions from 80 L of colostrum will be carried out as follows as described below.

Fat is removed from colostrum (80 L) by continuous flow centrifugation in a Westphalia apparatus (SA-1-02-175, GEA Mechanical Equipment US, Inc., Northvale, N.J.). The resulting defatted colostrum is diluted with 2 volumes of reverse osmosis water, and lactic acid is added to a final pH of 4.6 at 42° C. (DL-Lactic Acid, 85% solution, Fisher Scientific, Waltham, Mass.) to precipitate casein, with mixing by broad blade vertical impeller or equivalent mixing apparatus. Following the crude filtration or equivalent step such as cheese press to remove casein, the material is held overnight at 2-8° C. and then neutralized by Tromethamine addition (Trizma Base, Sigma Aldrich, St. Louis Mo.). Diatomaceous earth filter agent (Sigma Aldrich, St Louis, Mo.) is added to 4 g/mL prior to the first filter capsule with stirring for 10 min. The clarification filter train consists of a 20 μm Alpha fibrous polypropylene (Meissner Filtration Products, Camarillo, Calif.)/0.45 μm polypropylene filter CLMFO.45-222 (Meissner Filtration Products, Camarillo, Calif.)/0.2 μm filter (Pall Corporation, Port Washington, N.Y.). It will be recognized that other filter trains from these or other manufacturers will also equivalently prepare the sample for chromatography.

In this example, scale up is accomplished by dividing the sample into three aliquots and subjecting each portion to serial chromatography, with washing of the column set up in between samples. Capto-S resin (3 L bed volume) and Capto-Q resin (3 L bed volume) is packed in two INdEX 140/500 columns (GE Healthcare Bio-Sciences Corp., Piscataway, N.J.), connected in series. Prior to each sample load, the serial column set up is washed with 12 L reverse osmosis water, 12 L 0.5 M NaCl, 12 L reverse osmosis water, 12 L 1 M NaCl, 12 L reverse osmosis water, then 1 M Tris-HCl pH 6.8 (until pH is stabilized at 6.8). After the wash steps, the column is equilibrated with 18 L 10 mM Tris-HCl, pH 6.8. The pH and conductivity of the whey is measured and the whey is pumped onto the columns at a flow rate of 0.5 L/min, and the column set up is washed with 2.5 column volumes of equilibration buffer. Absorbance at 280 nm and pH will be monitored using an inline flow cell (PendoTECH, Princeton, N.J.). Collection of flow through is stopped when A280 approaches baseline levels. After chromatography, the pH and conductivity is measured and the pH is found to be within 0.2 pH units of the pH of the load material and the conductivity is found to be within 1 milliSiemens/cm of the load material. The product is concentrated by ultrafiltration (50 kDa NMWCO filter), using a Pall Pharmaceutical Series apparatus (Pall Corporation, Port Washington, N.Y.), then diafiltered versus 5 volumes of reverse osmosis water. The product is concentrated to >75 mg/mL by ultrafiltration. Terminal heat treatment is performed at 60° C. for 10 hours.

### Example 16

### Investigation by Mass Spectrometry of the Identity of Proteins in the Polyclonal Antibody Preparation

52 kg of colostral whey was loaded on to two 3 L columns of Capto-S and Capto-Q in series as described in Example 14. The flow through and strip fractions were analyzed by reducing SDS-PAGE and the relevant bands were excised from the gel. Samples were subjected to mass spectrometry. The analysis was performed on an LTQ-Orbitrap apparatus (Fisher ThermoScientific, Waltham, Mass.) at the University of Massachusetts. The resulting peptide sequences were used to search the NCBI nr database. Table 8 summarizes the most prevalent sequence for each band.

TABLE 8

| Band | | |
|---|---|---|
| number | Sample | Most prevalent sequences |
| 1 | Flow-through | IgG1 heavy chain |
| 2 | Flow-through | Transferrin, IgM, secretory component |
| | | (poly IgR) |
| 3 | Flow-through | IgG1 |
| 4 | Flow-through | IgG1 |
| 5 | Flow-through | Ig light chain (primarily lambda, some kappa) |
| 6 | Flow-through | Alpha-lactalbumin, keratin |
| 7 | Strip | Lactoferrin, transferrin, IgM, some |
| | | lactoperoxidase |
| 8 | Strip | Bovine serum albumin |
| 9 | Strip | Zinc alpha 2 glycoprotein, complement C3 |
| 10 | Strip | IgG1 (presumably fragments) |
| 11 | Strip | Beta-lactoglobulin |
| 12 | Strip | Pancreatic ribonuclease |
| 13 | Strip | Alpha-lactalbumin |
| 14 | Strip | Ig heavy chain, keratin |

An analysis of the flow-through material confirmed that the major bands on reducing SDS PAGE (bands 4 and 5) represent IgG heavy and light chains. The smearing above band 4 (band 3) is again IgG heavy chain and presumably represents different glycoforms. The high molecular weight band (band 1) seen in all analyses of bovine immunoglobulin is an aggregate of IgG heavy chain. A triplet of bands is seen in the sample labeled band 2. This triplet consists primarily of secretory component (79 kDa), IgM (76 kDa) and transferrin (73 kDa). Both secretory component and IgM are desired components of the composition, while transferrin is an impurity. The remaining low molecular weight band includes the impurities alpha-lactalbumin and keratin. These impurities will be removed during downstream polishing on ultrafiltration diafiltration.

An analysis of the material stripped from the columns confirmed that the process removed lactoferrin, bovine serum albumin, beta-lactoglobulin, and alpha-lactalbumin, as well as some immunoglobulin and some minor impurities.

This analysis showed that extraneous proteins that may confound production of a pharmacologically active polyclonal antibody preparation can be removed using this strategy, and that further polishing steps can be applied to produce compositions suitable for patient populations including those with compromised gastrointestinal systems.

### Example 17

### Direct Comparison of Compositions Purified Using Different Methods

A direct comparison was made of compositions of colostrum purified using four different methods: thioester T-gel chromatography (Example 2), ammonium sulfate precipitation (Example 4), MEP chromatography (Example 8) and Capto-S/Capto-Q serial chromatograph (Example 14). Samples of each preparation were analyzed by reducing SDS PAGE and by ELISA to quantify the levels of lactoferrin, alpha-lactalbumin, beta-lactoglobulin. Samples were also assayed by ELISA to quantify the levels of lactoperoxidase and IGF-1.

A reducing SDS PAGE analysis of these different compositions was prepared as follows: Lane 1: molecular weight markers; Lane 2: defatted pooled colostrum; Lane 3: Defatted, decaseinated whey; Lane 4, flow through Capto-S only; Lane 5, Flow through Capto-Q only; Lane 6, Flow through Capto-S/Capto-Q; Lane 7, MEP chromatography; Lane 8, ammonium sulfate-purified antibody preparation; Lane 9, T-gel-purified antibody preparation; Lane 10, affinity purified antibody specific for murine TNF. A densitometric analysis of the gel was conducted. These data confirm the results presented in the examples above and show that the four methods under investigation result in roughly comparable levels of purity as judged by this method (note that the T-gel and ammonium sulfate products were purified at the bench scale while the MEP and Capto-S/Capto-Q products were produced at pilot scale).

More significant differences were seen when assays were performed to quantify levels of specific impurities.

The samples were analyzed in the BCA assay to quantify total protein and by ELISA to quantify the levels of specific impurities. A commercially available ELISA kit (Cat. #E10-126, Bethyl Laboratories, Montgomery, Tex.) was used to quantify lactoferrin. Per manufacturer's recommendation, ELISA plates were coated with a 1:100 dilution of goat-anti bovine lactoferrin coating antibody reagent provided. The plates were washed, blocked, and serial dilutions of samples were added, washed, and binding detected with horseradish-peroxidase conjugated, affinity purified goat anti-bovine lactoferrin and 3,3′,5,5′-tetramethylbenzidine (TMB) as substrate. Commercially available ELISA kits (Cat. #E10-125 and #E10-128, Bethyl Laboratories, Montgomery, Tex.) were used to quantify beta-lactoglobulin and alpha-lactalbumin, respectively. Per the manufacturer's recommendation, ELISA plates were coated with a 1:100 dilution of the goat-anti bovine beta-lactoglobulin or alpha-lactalbumin coating antibody reagent provided. The plates were washed, blocked, and serial dilutions of samples were added, washed, and binding detected with horseradish-peroxidase conjugated, affinity purified goat anti-bovine beta-lactoglobulin or alpha-lactalbumin, respectively and 3,3′,5,5′-tetramethylbenzidine (TMB) as substrate. Commercially available ELISA kits (Cat.#KT-20283 and #KT-18278, Kamiya Biomedical Co., Seattle, Wash.) were used to determine the levels of bovine lactoperoxidase (LPO) and insulin-like growth factor I (IGF-I), respectively, in different preparations. Anti-bovine LPO or IGF-I antibodies are precoated on the 96-well strip plates provided. Serial dilutions of samples and calibrator standards were added and incubated prior to addition of detection reagent A. After additional incubation, wells were washed and detection reagent B added and incubated. Finally, wells were washed and incubated with 3,3′,5,5′-tetramethylbenzidine (TMB) substrate solution, followed by stop solution prior to being read at 450 nm. Tables 9 and 10 summarize the results.

TABLE 9

| | | | Beta- |
|---|---|---|---|
| | Lactoferrin | Alpha-lactalbumin | lactoglobulin |
| Sample | mg/g | mg/g | mg/g |
| Defatted colostrum | 10 | 88 | 197 |
| Whey | 11 | 146 | 70 |
| Capto-S/Capto-Q | 0.3 | 75 | 0.5 |
| MEP | 30 | 4.0 | 7.6 |
| Ammonium sulfate | 2.4 | >20 | 25 |
| T-gel | 0.5 | ND | ND |

TABLE 10

| | IGF-1 | Lactoperoxidase |
|---|---|---|
| Sample | mg/g | mg/g |
| Whey | >5.1 | 64 |
| Capto-S/Capto-Q 8Feb | 0.09 | 0.18 |
| Capto-S/Capto-Q 10Feb | 0.05 | 0.21 |
| MEP | 41 | 21 |
| T-gel | 0.12 | 1.8 |

### Example 18

### Reducing SDS PAGE Analysis of Composition Purified on Capto-S/Capto-Q Chromatography Followed by Ultrafiltration

Immunoglobulin was purified from colostral whey as described in Example 14. The material that flowed through the serial Capto-S and Capto-Q columns was subjected to ultrafiltration on a 30,000 molecular weight cut-off membrane and the retentate was analyzed by reducing SDS-PAGE and densitometric analysis of the gel.

A comparison of the data in this example with that in Example 17 demonstrates that the addition of the ultrafiltration step cleanly removes the alpha-lactalbumin remaining in the Capto-S/Capto-Q flow through. The material analyzed in Example 17 had to a peak area of alpha-lactalbumin of 1% in the densitometry analysis which corresponded to a concentration of 75 mg/g of alpha-lactalbumin by ELISA (see Example 17). Following ultrafiltration, there was no alpha-lactalbumin detectable on the SDS-PAGE analysis, indicating that the level of alpha-lactalbumin is <15 mg/g.

Based on densitometry, this composition is 97% immunoglobulin: 55% Ig heavy chain (IgG and IgA), 33% Ig light chain (kappa and lambda), 3% secretory IgM heavy chain and an impurity of 3% transferrin.

Capto-Q is a strong anion exchanger and Capto-S is a strong cation exchanger. Typically one would optimize the pH to bind one resin or the other, based on the pI of the protein. However, polyclonal antibodies have a broad pI range, complicating this approach. A novel approach to using these columns such that the highest yield of purified and isolated immunoglobulin could be achieved, was to choose a pH in the middle of the predicted pI range for the polyclonal immunoglobulin, such as a pH in the range of 6.6 to 7.0 as shown in Table 11.

TABLE 11

| | pI | pH 7 | Should | pH 5.8 | Should |
|---|---|---|---|---|---|
| Protein | value | charge | Bind | charge | Bind |
| b-lactoglobulin | 5.2-5.4 | negative | Q (+) | mostly | nothing |
| | | | | neutral | |
| a-lactalbumin | 4.3-5.1 | negative | Q (+) | neutral to | weakly to |
| | | | | negative | Q (+) |
| polyclonal bovine | 5.8-7.3 | neutral to | weakly | neutral to | weakly to |
| immunoglobulin | | positive | to S (−) | negative | Q (+) |
| population | | | | | |
| lactoferrin | 7.8-8.0 | weakly | weakly | positive | S (−) |
| | | positive | to S (−) | | |
| lactoperoxidase | 9.2-9.9 | positive | S (−) | strongly | S (−) |
| | | | | positive | |
| BSA* | 5.13 | negative | Q (+) | mostly | nothing |
| | | | | neutral | |

The experiments described herein determined that it was preferable to use a flow-through approach rather than bind and elute as the flow through provides faster throughput, less use of expensive buffers, and resulted in a more highly purified preparation. If the conditions are not correct, then some immunoglobulin will bind to the resin, resulting in reduced yields. The novel approach described herein optimized the conditions that resulted in the highest yield with the highest purity of immunoglobulin composition

### Example 19

### Increase in Potency Through Purification of Immunoglobulin

A) Polyclonal anti-hTNF antibody in accordance with the invention also referred to herein as “AVX-470” was prepared and purified using methods similar to those described in Examples 3 and 14. Colostrum was defatted by centrifugation and assayed for the presence of anti-TNF antibody by ELISA. Protein concentration was determined by BCA and the activity of the defatted colostrum was expressed as AU/mg of protein. In two separate runs, the defatted colostrum was found to have 338 AU/mg and 277 AU/mg. The defatted colostrum was further purified by acid precipitation of casein, filtration, serial anion and cation exchange chromatography, diafiltration and ultrafiltration and heat treatment. The final samples were reassayed for anti-TNF activity by ELISA and protein concentration by BCA. The final drug substance lots were found to have 634 and 526 AU/mg protein. Therefore, throughout the purification steps, the potency was increased by 1.9 fold in each lot, consistent with the enrichment of immunoglobulin.

B) Affinity Purification of AVX-470

An affinity matrix was prepared by coupling recombinant human TNF to Affigel-10. Briefly, 3 mg human TNF (Cell Sciences, Catalog number CRT100C, lot 3105816), prepared as a lyophilized powder from PBS (phosphate-buffered saline, pH 7.2) was reconstituted and combined with 0.5 mL Affigel-10 (BioRad) for coupling, followed by washing, blocking and storage according to the manufacturer's instructions. AVX-470 as prepared in (A) above, was diluted in PBS and passed over the column. The matrix was washed with 20-column volumes of PBS, and eluted with 2.5-volumes 50 mM citric acid/100 mM sodium chloride, pH 2.0 with collection into vessels containing sufficient 1M Trizma base solution to provide effectively immediate neutralization. Samples were analyzed by absorbance at 280 nm and initially evaluated based on a rough conversion factor of 1.4 mg/mL-A_{280 nm}. A standard in vitro L929 assay for hTNF-induced cytotoxicity showed a 311-fold purification of TNF neutralizing activity of the affinity purified material designated as “AVX-470A”. The data indicate that 0.3% of AVX-470A is specific for TNF. The potency of the affinity purified antibody is very similar to that of infliximab (EC50=51 ng/ml vs 76 ng/ml for infliximab) as shown in Table 12.

TABLE 12

| | EC50 in |
|---|---|
| | TNF |
| | ELISA |
| Antibodies | ug/ml |
| AVX-470 | 15.9 |
| Infliximab | 0.076 |
| AVX-470A | 0.051 |

### Example 20

### Immunization of Cows with Recombinant Human TNF

Twelve male dairy calves, ages 3 to 5 months, were selected for the immunization study. Following a three week quarantine period, each calf was immunized four times with 0.05 mg recombinant human TNF (rhTNF) combined with one of four possible adjuvants: Quil A (0.5 mg/mL), Montanide ISA 201 VG (50% solution), EMULSIGEN®-D (20% solution) or EMULSIGEN®-BCL (20% solution).

The rhTNF was supplied as a lyophilized powder by Cell Sciences and prepared up to 2 days in advance of use as a 0.05 mg/mL solution in 1 mg/mL bovine serum albumin. Quil A adjuvant was supplied as a lyophilized powder by Accurate Chemical & Scientific Corp. and a 1 mg/ml solution was prepared on the day of use. Montanide ISA 201 VG was supplied as a ready-to-use liquid by Seppic, Inc. EMULSIGEN®-D and EMULSIGEN®-BCL adjuvants were supplied as ready-to-use liquid by MVP Technologies, Inc.

There were three calves per adjuvant group. Each immunization was 2 cc in volume and all were administered subcutaneously in the neck or shoulder region. The day of the first immunization was designated Study Day 0; subsequent immunizations were administered on Days 21, 35 and 56. Animals were observed for 72 hours after each immunization and any abnormal or unusual findings were reported. Fifty mL serum samples were collected on Days 0 (pre-immunization bleed), 21, 35, and 56, and a final large volume (300-500 mL) sample was collected on Day 70 (two weeks after the final immunization) for immunologic analyses.

Serum samples were obtained by collection of whole blood from the jugular vein. The final large volume bleed was collected into 250 mL sterile collection bags.

The serum-derived material produced by immunization of the calves has been named Serum-470.

### Example 21

### Binding to Human TNF by ELISA

To determine whether Serum-470 could bind to human TNF, serum samples were assayed by ELISA. Pools were created from each of the groups of animals immunized with a given adjuvant, using the fourth bleed sample. Recombinant human TNF (Cell Sciences) was diluted into 0.05M carbonate buffer (pH 9.6) and coated onto 96-well plates, 0.1 mL/well, at a concentration of 1 ug/ml. After a 1-hour incubation at room temperature, plates were washed five times with 0.05% Tween in 0.01 M Tris-buffered saline (TTBS). Serum samples were diluted in TTBS and 0.1 mL was added to duplicate wells in a 3-fold dilution series from 1:10-1:590,490. After one hour at room temperature, plates were washed five times with TTBS, and 0.1 mL horseradish peroxidase-conjugated sheep anti-bovine IgG (h+1) (Bethyl Labs, Montgomery Tex.) was added to each well at a dilution of 1:100,000.

After one hour at room temperature, plates were washed five times with TTBS, and colorimetric analysis was performed after the addition of 50 μl/well 3,3′,5,5′ tetramethyl benzidine (TMB) substrate (Invitrogen, Carlsbad, Calif.) incubated for thirty minutes in the dark, followed by 0.05 mL/well 1% H₂SO₄ stop solution.

Optical densities were determined at 450 nm. Background absorbance, determined from wells without serum, was subtracted from the experimental OD 450 values.

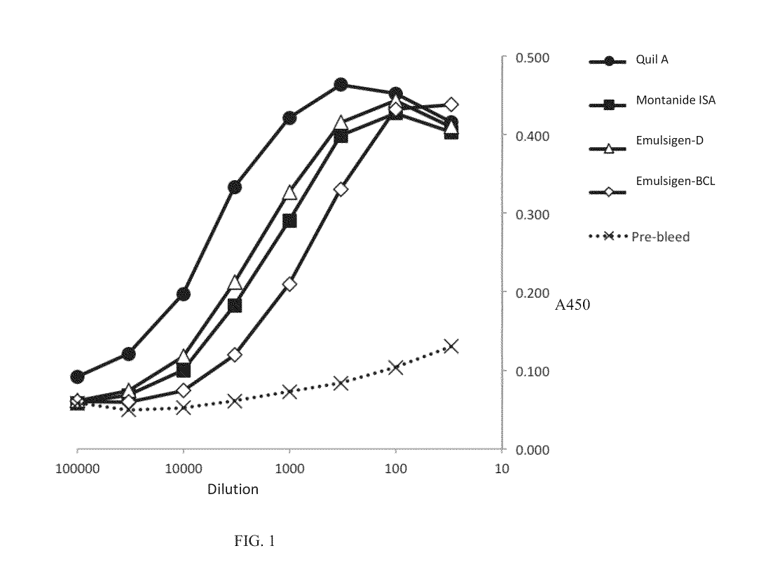

FIG. 1 shows the anti-TNF ELISA data. All four groups of immunized animals had antibodies that bound to human TNF, although the titer of the anti-TNF antibodies differed between groups, with Quil A being the most effective adjuvant. Little if any antibody was detected in the pre-bleed serum sample.

To determine whether the difference in titers might be due to a change in the total immunoglobulin concentration in the different groups, the serum samples were assayed for total immunoglobulin concentration by ELISA. Microtiter plates were coated as above with anti-bovine IgG (h+1) antibody (Bethyl Laboratories) at 5 ug/ml. Pools of Serum-470 were serially diluted and added to the plates and washed. Binding was detected using horseradish peroxidase-conjugated sheep anti-bovine IgG antibody.

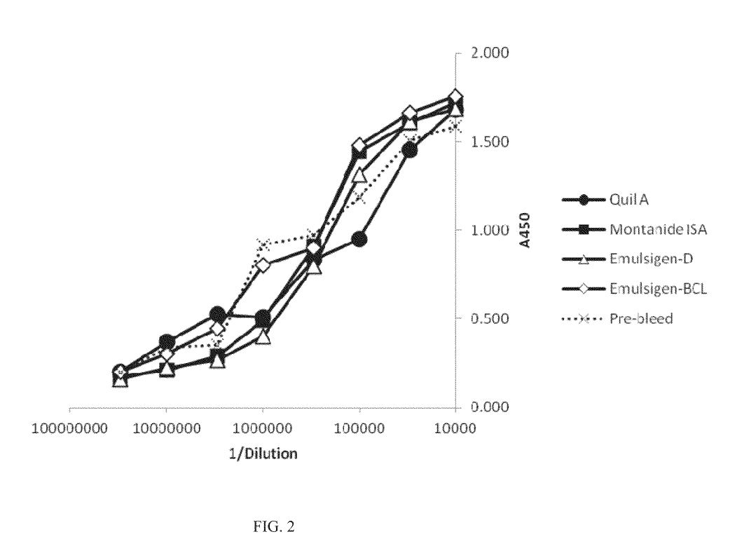

FIG. 2 shows the anti-Ig ELISA data. All four groups and the pre-bleed had comparable concentrations of immunoglobulin. Therefore, the difference in anti-TNF titer between groups seen in FIG. 1 represented differences in the percentage of the immunoglobulin that was specific for human TNF.

### Example 22

### Neutralization of Human TNF by Serum-470

The ability of the Serum-470 samples (pooled samples from bleed 4) to neutralize human TNF was determined using a standard TNF cytotoxicity bioassay (Mathews N., et al 1987, Lymphokines and Interferons) using the murine L929 fibroblast cell line (American Type Culture Collection, Rockville, Md.; Cat# CCL-1). L929 cells were maintained by serial passage in culture medium comprised of Eagle's Minimal Essential Medium (EMEM) containing 10% fetal bovine serum (Invitrogen, Carlsbad, Calif.). The day before the assay, L929 cells were harvested from the tissue culture flasks by brief trypsinization with 0.25% trypsin-EDTA (Invitrogen). The cells were washed in culture medium and 6×10⁴ cells/well were dispensed in 0.1 mL aliquots into a 96-well plate and incubated in a 37° C. CO₂ incubator overnight. Serial 3-fold dilutions (0.06 mL/well) of Serum-470 samples in culture medium containing 2 ug/ml actinomycin-D (Sigma-Aldrich, St Louis, Mo.) were prepared in duplicate wells in a 96-well microtiter plate.

Recombinant human TNF (rhTNF; Cell Sciences In., Canton; MA; Cat#CRT-100C) (0.06 μL/well at 4 ng/ml in culture medium containing 2 ug/ml actinomycin-D) was added to each well and the mixtures were preincubated for 1 hr at 37° C. The Serum-470/rhTNF mixtures (0.01 mL/well) were added to the confluent cultures of L929 cells and incubated at 37° C. for 20 hr. The final volume per well is 0.2 mL containing 1 ng/ml rhTNF, 1 ug/ml actinomycin-D, and eight 3-fold dilutions of Serum-470 samples starting at 1:30.

Control wells included medium plus cells or rhTNF plus cells. Viability of the L929 cells was assessed by adding 0.02 mL of Promega Substrate: CellTiter 96 Aqueous One Solution Reagent (Promega, Madison, Wis.) to each well, incubating the plates for 6 hours in a 37° C. CO₂ incubator and reading the OD in each well at 490 nm in an ELISA plate reader. Potency of TNF neutralization was calculated as an IC₅₀ value of serial dilutions of the Serum-470 samples in the presence of a fixed quantity (1 ng/ml) of TNF. Data are expressed as the reciprocal of the dilution that lead to a half-maximal inhibition of TNF activity.

As shown in Table 13, all four adjuvants induced antibody that could neutralize human TNF, while no activity was seen in the pre-bleed. Quantitative differences were seen between the adjuvants, with Quil A and Montanide ISA 291 VG being the most effective at inducing a strong neutralizing antibody response.

TABLE 13

| Sample | IC₅₀ |
|---|---|
| Quil A | 8300 |
| Montanide ISA 291 VG | 8300 |
| Emulsigen-D | 2200 |
| Emulsigen-BCl | 1300 |
| Pre-bleed | No activity |

### Example 23

### Species specificity of Serum-470 (Quil A)

The TNF species specificity of Serum-470 was determined using a TNF ELISA. Recombinant TNF (rTNF) from 9 species were tested (human, canine, cynomolgus monkey, rhesus macaque, guinea pig, porcine, murine, rat, bovine). TNF samples were purchased from Cell Sciences (human, murine), R&D Systems (canine, rhesus macaque, guinea pig, porcine, rat, bovine) or Sin θ Biological, Inc. (cynomolgus monkey). Microtiter plates were coated with rTNF from the different species and assayed as described in Example 2. Serum samples were a pool from calves immunized with rhTNF and Quil A adjuvant. ELISA titers were determined using nonlinear regression analysis.

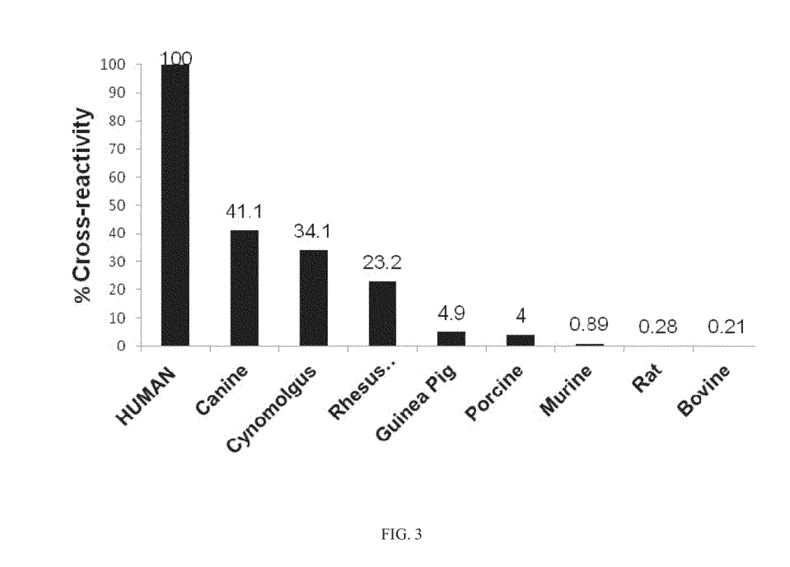

FIG. 3 shows the relative titers of Serum-470 (Quil A) for TNF from different animal species.

### Example 24

### Effect of Adjuvant on Antigenic Specificity

To determine whether the adjuvant used in the immunization might affect the specificity of the induced antibody, pools from each of the groups of calves were assayed for their ability to bind to human or canine TNF by ELISA. Assays were run as in Examples 21 and 23.

Table 14 shows the relative binding to human and canine TNF by ELISA. All four serum pools had a higher titer when assayed on human TNF than when assayed on canine TNF. However, the relative titers varied between adjuvants. Serum from animals immunized with Quil A, Montanide ISA 201 VG, or Emulsigen BCL, all had relative titers of 2.2-2.5. However, serum from animals immunized with Emulsigen D displayed a larger degree of species specificity, with a 5.5-fold preference for human TNF over canine TNF.

These data demonstrate that the choice of immunization conditions including the adjuvant affects the specificity profile of the antibody.

TABLE 14
Relative Titers as Determined by ELISA

| | | Montanide | | Emulsigen- |
|---|---|---|---|---|
| | Quil A | ISA | Emulsigen D | BCL |
| Human rTNF | 6425 | 1437 | 3275 | 685 |
| Canine rTNF | 2604 | 599 | 593 | 312 |
| Ratio | 2.47 | 2.40 | 5.52 | 2.20 |

### Example 25

### Species Specificity Profile of Serum-470: Comparison of ELISA Binding Data and L929 Neutralization Data

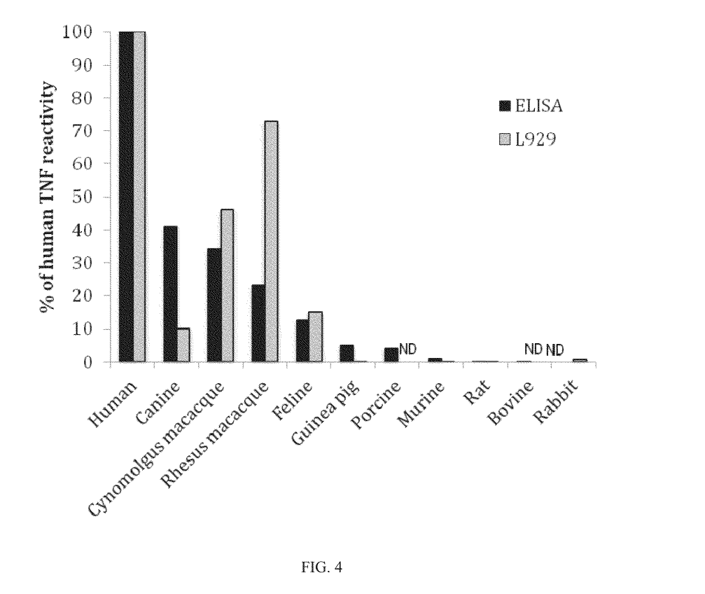

The species specificity profile of Serum-470 (Quil A) by ELISA was determined as described in Example 24 and the data shown in FIG. 4 are identical to those described in Example 24. The species specificity profile of Serum-470 using the L929 neutralization assay were determined in a similar manner to the data shown in Example 22, except that TNF from different species were used in the assay. Each species of TNF was assayed at the approximate ED90 (the concentration at which the L929 readout was reduced by 90%). K is were calculated using the equation Ki=IC50/[1+(A/ED50)] where IC50=the dilution of AVX-470 giving 50% inhibition of the response; A=the concentration of TNF used in the assay; and ED50=the half-maximal concentration of TNF from the particular species needed to inhibit L929 cells.

To also calculate a concentration-based IC₅₀ for each TNF, it was assumed that 1% of the total immunoglobulin concentration for the Serum-470 (Quil A) (8 mg/mL; FIG. 12) was TNF-specific (i.e. 0.08 mg/mL of undiluted serum). The undiluted concentration of TNF-specific antibody (0.08 mg/mL) was then divided by the Dilution IC₅₀ and substituted into the Cheng-Prusoff equation, resulting in Kᵢ expressed as both as mg/mL and as pM (Table 15). The Kᵢ of 3.47 pM for human TNF is comparable to the Kᵢ of 4 pM reported for the sheep anti-human TNF antibody fragment AZD9773 reported by AstraZeneca (Newham et al., 2011).

The data shown in Table 15 and FIG. 4 demonstrate the species specificity profile of the serum-derived AVX-470 polyclonal antibody.

TABLE 15

| | | | A (pg/ | ED₅₀ | Kᵢ | Kᵢ |
|---|---|---|---|---|---|---|
| TNF species | Dilution IC₅₀ | IC₅₀* | mL) | (pg/mL) | (mg/mL) | (pM)** |
| Human | 5.00E−05 | 4.00E−06 | 400 | 60 | 5.22E−07 | 3.47 |
| Rhesus | 1.10E−04 | 8.80E−06 | 2000 | 175 | 7.08E−07 | 4.72 |
| macaque | | | | | | |
| Cynomolgus | 1.70E−04 | 1.36E−05 | 2000 | 175 | 1.09E−06 | 7.27 |
| monkey | | | | | | |
| Feline | 4.00E−04 | 3.20E−05 | 30000 | 5000 | 4.57E−06 | 30.47 |
| Canine | 1.00E−03 | 8.00E−05 | 30000 | 5000 | 1.14E−05 | 76.00 |
| Rabbit | >3.70E−03 | >2.96E−04 | 400 | 35 | >2.38E−05 | >158.67 |
| Murine | >3.70E−03 | >2.96E−04 | 2000 | 200 | >2.69E−05 | >179.33 |
| Guinea pig | >3.70E−03 | >2.96E−04 | 400 | 45 | >3.00E−05 | >200.00 |
| Rat | >3.70E−03 | >2.96E−04 | 100 | 100 | >1.48E−04 | >986.67 |
| Bovine | >3.70E−03 | >2.96E−04 | 10000 | 500000 | >2.90E−04 | >1933.33 |
| Procine | >3.70E−03 | >2.96E−04 | 10000 | 500000 | >2.90E−04 | >1933.33 |

*IC₅₀ value derived using total serum Ig quantitation (110811) of 8 mg/mL and assuming 1% is TNF-specific: (8 mg/mL)(0.01)/(semm dilution that gave 50% inhibition of cell death in L929 assay)
**Assuming all antibodies are 150000 Da

The Kᵢ values for various species of TNF can be compared to the Kᵢ for human TNF based on inverse Kᵢ comparison (as the smaller Kᵢ indicates a higher inhibition potency). This analysis gives a quantitative measure of the degree of cross-reactivity of Serum-470 towards TNF from different species relative to human TNF. Compared to human TNF, Serum-470 shows the greatest neutralization cross-reactivity for rhesus macaque TNF (73%) and cynomolgus monkey TNF (48%).

A summary relating the relative fold differences from ELISA (fold reduction) and L929 Kᵢ (fold increase) is in Table 16.

TABLE 16
ELISA titer and L929 Kᵢ summary (relative fold activity)

| TNF | ELISA titer fold reduction | Ki fold increase |
|---|---|---|
| Human | (—) | (—) |
| Rhesus macaque | 4.3 | 1.4 |
| Cynomolgus monkey | 2.9 | 2.1 |
| Feline | 10.5 | 8.9 |
| Canine | 2.4 | 21.9 |
| Rabbit | ND | >45.7 |
| Murine | 112.1 | >51.7 |
| Guinea pig | 20.4 | >57.6 |
| Rat | 361.3 | >284.3 |
| Bovine | 465.3 | >557.2 |
| Porcine | 25.0 | >557.2 |

The potency assays using Serum-470 suggest that the highest degree of binding and neutralization cross-reactivity of AVX-470 will likely be with the non-human primates, rhesus macaque and cynomolgus monkey from among the 10 species evaluated in the present studies.

As highlighted in FIG. 4 the data further demonstrate that the binding and neutralization data do not correlate and that it is critical to measure both.

### Example 26

### Colostrum Derived AVX-470 Antibody Shows the Same Species Specificity as Serum 470

a) Differences in species specificity of AVX-470 as prepared in Examples 3 and 14 from colostrum was measured in anti-TNF ELISA (binding).

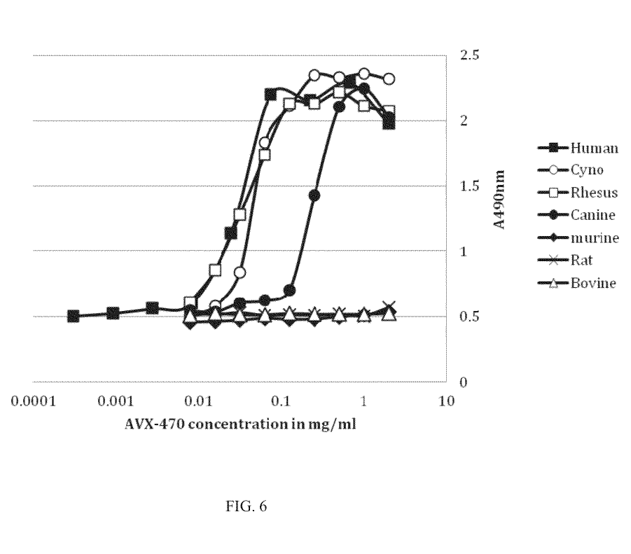

TNF from varying species was purchased from commercial suppliers. Human and murine TNF were from Cell Sciences, rhesus macaque, canine, and bovine were from R&D Systems, and cynomolgus monkey was from Sino Biological, Inc. ELISA plates (Greiner Bio-One) were coated with TNF from varying species at 1 μg/mL in 0.1 mL carbonate coating buffer, pH 9.6 (SIGMA#C3041), washed and blocked with 0.05% TWEEN in Tris-buffered saline (TBS-TWEEN). Serial dilutions of AVX-470 were added and incubated for 1 hour at room temperature. Plates were developed with horseradish peroxidase (HRP)-conjugated sheep anti-bovine antibody (Bethyl Laboratories) and 3,3′,5,5′ tetramethylbenzidine substrate (Invitrogen) and stopped by the addition of 1% H₂SO₄. Background values for each TNF species incubated without serum were subtracted from each serum-containing well. Data are shown in FIG. 6. According to the data, the species specificity of colostrum derived AVX-470 antibody is similar to that from AVX-470 antibody derived from serum indicating that the antibodies derived from either the colostrum or the serum of a bovine that has been immunized in accordance with the process described in Example 3 are the same.

b) Species specificity of AVX-470 as measured in L929 assay (neutralization).

The ability of AVX-470 antibody as prepared in accordance with Examples 3 and 14 to neutralize TNF was determined using the murine fibroblast cell line. The effective TNF concentration that killed 90% of L929 cells (EC₉₀) was first calculated for each species' TNF in order to begin anti-TNF antibody neutralization experiments at the top of the linear portion of the sigmoid dose response curve. TNF from varying species was purchased from commercial suppliers. Human and murine TNF were from Cell Sciences, rhesus macaque, canine, and bovine were from R&D Systems, and cynomolgus monkey was from Sino Biological, Inc. L929 cells were cultured overnight at 3.5×10⁴ cells/well in a 96 well plate and then incubated for 20 hr with serial dilutions of TNF in 1 μg/mL of actinomycin D. Cell viability was assessed by a 4 hour culture with CellTiter 96 Aqueous One Solution (Promega, Madison, Wis.). The EC₉₀ for each species' TNF was calculated using 0.72 OD at 490 nm as this correlated with ˜90% cell death caused by most species' TNF. To assess neutralization by AVX-470, L929 cells were seeded as above. Serial dilutions of AVX-470 were pre-incubated with recombinant TNF (at EC₉₀) for 1 hour at 37° C. before being added to the L929 cells with actinomycin D. Cell viability was assessed using the Promega Substrate per the TNF titration assay. The EC₅₀ is the concentration of AVX-470 that resulting in 50% inhibition of TNF-mediated killing of L929 cells and is shown in Table 17.

TABLE 17

| Source of TNF | EC50 (mg/ml) |
|---|---|
| Human | 0.028 |
| Rhesus macaque | 0.032 |
| Cynomolgus monkey | 0.048 |
| Canine | 0.25 |
| Murine | >2.0 |
| Rat | >2.0 |
| Bovine | >2.0 |

As was shown in Example 26(a) above, the TNF neutralizations profile for the serum derived serum-470 is the same as that for the colostrum derived AVX-470. The data also confirm that the fine specificity for species cross reactivity.

### Example 27

### Epitope Mapping

a) Summary A sample of AVX-470 prepared by methods similar to those described in Examples 3 and 14, was obtained comprising anti-hTNF polyclonal antibodies derived from the colostrum of cows immunized with rhTNF in accordance with the immunization scheme of Example 3. Based on CLIPS technology described in Timmerman et al. (2007) J. Mol. Recognit., 20:283-99, a total of 2625 different peptides were designed to reconstruct possible conformational and discontinuous epitopes of human TNF wherein sequence of TNF used for epitope mapping has the amino acid sequence of amino acids 1-157 of SEQ ID NO: 1:

(SEQ ID NO: 1)
VRSSSRTPSDKPVAHVVANPQAEGQLQWLNRRANALLANGVELRDNQLVV
PSEGLYLIYSQVLFKGQGCPSTHVLLTHTISRIAVSYQTKVNLLSAIKSP
CQRETPEGAE AKPWYEPIYLGGVFQLEKGDRLSAEINRPDYLDFAESGQ
VYFGIIAL

It should be noted that SEQ ID NO: 1 does not include the cytoplasmic and transmembrane regions of hTNF. It is understood that the amino acid numbers may differ in the full length sequence for hTNF.

The binding of antibodies of sample AVX-470 to each synthesized peptide was tested using ELISA. ELISA analysis of sample AVX-470 onto peptides of hTNF, using different concentrations of sample and multiple variations of blocking conditions identified five distinct binding regions (epitopes) of TNF:

(SEQ ID NO: 2)
₁VRSSSRTPSDKPVAH₁₅
(SEQ ID NO: 3)
₂₁QAEGQLQWLNRRANA₃₅
(SEQ ID NO: 4)
₆₁QVLFK₆₅
(SEQ ID NO: 5)
₉₁VNLLS₉₅
(SEQ ID NO: 6)
₁₃₁RLSAEINRPD₁₄₀

Projection of the epitopes onto the relevant crystal structure of hTNF shows that the epitopes of SEQ ID NO: 2, SEQ ID NO: 3, and SEQ ID NO: 5 are surface exposed. The epitope of SEQ ID NO: 6 is surface exposed, but is mostly in contact with another TNF monomer in the trimer state. The epitope of SEQ ID NO: 4 is mostly buried inside the TNF monomer.

b) Methods—Synthesis of Peptides and Screening Procedures

To reconstruct discontinuous epitopes of the target molecule, a library of structured peptides was synthesized. This was done using Chemically Linked Peptides on Scaffolds (CLIPS) technology described in Timmerman et al., (2007) J. Mol Recognit. 20:283-99. CLIPS technology allows to structure peptides into single loops, double-loops, triple loops, sheet-like folds, helix-like folds and combinations thereof. CLIPS templates are coupled to cysteine residues. The side-chains of multiple cysteines in the peptides are coupled to one or two CLIPS templates. For example, a 0.5 mM solution of the T2 CLIPS template 1,3-bis(bromomethyl) benzene is dissolved in ammonium bicarbonate (20 mM, pH 7.9)/acetonitrile (1:1(v/v). This solution is added onto the peptide arrays. The CLIPS template will bind to side-chains of two cysteines as present in the solid-phase bound peptides of the peptide-arrays (455 wells plate with 3 ul wells as described in Sloostra et al., (1996) Molecular Diversity 1:87-96). The peptide arrays are gently shaken in the solution for 30 to 60 minutes while completely covered in solution. Finally, the peptide arrays are washed extensively with excess of H₂O and sonicated in disrupt-buffer containing 1 percent SDS/0.1 percent beta-mercaptoethanol in PBS (pH 7.2) at 70° C. for 30 minutes, followed by sonication in H₂O for another 45 minutes. The T3 CLIPS carrying peptides were made in a similar way but now with three cysteines.

The binding of antibody to each synthesized peptide was tested in an ELISA. The peptide arrays were pre-incubated for 30 minutes at room temperature with 5% blocking solution (the blocking solution consists of 4% ovalbumin, 5% horse sample, 1% Tween 80). The peptide arrays were incubated with primary antibody solution (1 to 100 ug/ml in PBS/1% Tween 80, overnight at 4° C.). After washing, the peptide arrays were incubated with a rabbit-anti-sheep antibody ( 1/1000, one hour at 25° C.), and after washing, a swine-anti-rabbit antibody peroxidase conjugate ( 1/1000, on hour at 25° C.). After washing, the peroxidase substrate 2,2′-azino-di-3-ethylbenzthiaxoline sulfonate (ABTS) and 2 microliters/milliliter of 3 percent H₂O₂ were added. After one hour, the color development was measured. The color development was quantified with a charge coupled device (CCD)—camera and an image processing system.

c) Results

1. Global Results and Signal-to-Noise Ratios

The binding activity of all peptides were analyzed using four different concentrations of AVX-470. Of the 25 ug/ml concentration, four different blocking conditions were applied. Together these 7 screenings are optimal to identify binding regions on the target protein and to discern stronger and weaker binding regions.

2. Identification of Epitopes

All data sets generated for this analysis have been visualized in linear plots as “segment” plots, or as heatmaps. At the lowest experimental concentration of applied sample, dominant binding was observed by peptides in the N-terminal region of the target protein (VRSSSRTPSDKPVAH) (SEQ ID NO: 1) the 5 high bars at approx. 1000 on the most left side of the graph. This finding was consistent over all experimental peptide groups. When binding conditions are altered, either by manipulating levels of sample or experimental blocking conditions, it can be observed that multiple independent binding regions may be identified. Heatmap visualization showed binding to

(SEQ ID NO: 2)
VRSSSRTPSDKPVAH
(SEQ ID NO: 3)
QAEGQLQWLNRRANA
(SEQ ID NO: 4)
QVLFK
(SEQ ID NO: 5)
VNLLS

At high concentrations of sample and low blocking strength some signal is also observed for residues (RLSAEINRPD) (SEQ ID NO: 6).

### Discussion:

VRSSSRTPSDKPVAH (SEQ ID NO: 2) is a dominant epitope and is involved in polyclonal antibody binding, but not necessarily neutralization of activity. The potential that the binding of this epitope by the polyclonal antibody of the invention results in neutralization is low because the sequence of residues 3-15 of canine and feline TNF is almost identical to human TNF (canine: VKSSSRTPSDKPVAH (SEQ ID NO: 7); feline: LRSSSRTPSDKPVAH (SEQ ID NO: 8)), and neutralization of canine and feline TNF by the polyclonal antibodies of the invention is less than 20% as per FIG. 4. However, Dong et al. have recently demonstrated that antibodies specific for sequence 80-91 (SSRTPSDKPVAH (SEQ ID NO: 9)) can inhibit collagen-induced arthritis in animal models and have designated this as a novel neutralizing epitope (Dong et al PLoS ONE (2010) 5:e8920).

QAEGQLQWLNRRANA (SEQ ID NO: 3) is surface exposed and partially overlaps with one of the binding sites of etanercept (amino acids 29-36 of SEQ ID NO: 1), a recombinant human soluble TNF receptor antagonist that is a dimeric fusion protein generated by linking the extracellular domains of human TNFR2 to the FC portion of human IgG1 (sold as Enbrel® by Amgen of Thousand Oaks, Calif.). This epitope is likely to be relevant in binding and neutralization of hTNF by the polyclonal antibodies of the invention.

QVLFK (SEQ ID NO: 4) is buried in the monomer and is not likely to be relevant for binding or neutralization.

VNLLS (SEQ ID NO: 5) is surface exposed and partially overlaps with one of the binding sites of etanercept which is the equivalent to amino acids 83-91 of SEQ ID NO: 1. However, this region is highly conserved (identical sequence found in canine, feline, pig, mouse, and rabbit) and is not likely to be relevant in neutralization of hTNF by the polyclonal antibodies of the invention.

RLSAEINRPD (SEQ ID NO: 6) appears to be a weak binder. The epitope is surface exposed on the hTNF monomer is in contact with another monomer in the hTNF trimer. There is partial overlap with an epitope of infliximab (a chimeric monoclonal anti-hTNF antibody sold as Remicade® in the U.S. by Janssen Biotech Inc.) There are several surprising aspects of this epitope. First, the epitope is in a highly conserved region between human TNF and bovine TNF. Thus, it would not be expected to be immunogenic in a bovine, which is the source animal of the polyclonal antibodies of the invention. However, it is likely to play a role in binding and neutralization as per Example 30 which shows that the polyclonal antibodies AVX-470 are as potent as infliximab in neutralizing hTNF in neutralization assay.

Polyclonal antisera induced by immunization of animals with human TNF and an appropriate adjuvant has been described using mice (Corti et al, Molecular Immunology (1992) 29:471-479, rabbits (Corti, 1992) and goats (Yonei et al., J. Biol. Chem. (1995) 270:19509-19515). Corti (1992) showed that anti-hTNF antibodies derived from mouse serum recognized epitopes 1-23, 95-116, 117 to 136 and 137-157 of hTNF as shown in SEQ ID NO: 1. Corti (1992) also showed that anti-hTNF antibodies derived from rabbit recognized epitopes 1-23, 56-75, 95-116, 137-157 of hTNF as shown in SEQ ID NO: 1. Of these epitope 1-23 was the most immunogenic. Yonei (1995) showed that anti-hTNF antibodies derived from goat recognized epitopes 7-11, 17-23, 30-39, 42-49, 106-112, 135-142 of hTNF as shown in SEQ ID NO: 1.

When compared to the AVX-470 bovine derived antibodies disclosed in this example, it is clear that immunizing cows with hTNF under the conditions described here results in a unique immunodominant footprint. Although different techniques were used in each of these studies to define the immunogenic epitopes, it is clear that the pattern seen in AVX-470 is distinct from that seen in the other reports. Notably, antibody responses to 61-65 and 91-95 of AVX-470 are not reported in any of the other species. In addition, the immunodominant site reported in mouse serum (104-112) was also detected in rabbit serum (95-116) and goat serum (106-112) but was not seen with AVX-470.

### Example 28

### Affinity of AVX-470 for TNF as Measured by a Competition ELISA

96-well flat bottomed NUNC MaxiSorp ELISA plates were coated overnight with 35 ng/ml recombinant human TNF (Cell Sciences) in carbonate coating buffer. Plates were washed with PBS-T (PBS containing 0.05% Tween-20) and blocked with 1% bovine serum albumin in PBS-T. AVX-470A (affinity purified AVX-470 purified as described in Example 19(b)) and infliximab (humanized anti-TNF monoclonal antibody used as a positive control) were incubated for 1 hour at room temperature with varying concentrations of TNF. The concentrations of AVX-470A and infliximab were 35 ng/ml and 2 ng/ml, respectively; these concentrations were selected because they were in the linear portion of the antibody dose response curve in a direct binding ELISA. The TNF-antibody mixtures were transferred to the TNF coated plates and incubated for 2 hours at room temperature. Plates were washed and antibody binding was detected using HRP-labeled sheep anti-bovine antibody (Bethyl Labs) for the AVX-470 samples or HRP-labeled mouse anti-human antibody (Abcam) for the infliximab samples. Plates were developed with TMB substrate and stopped with 1% H₂SO₄.

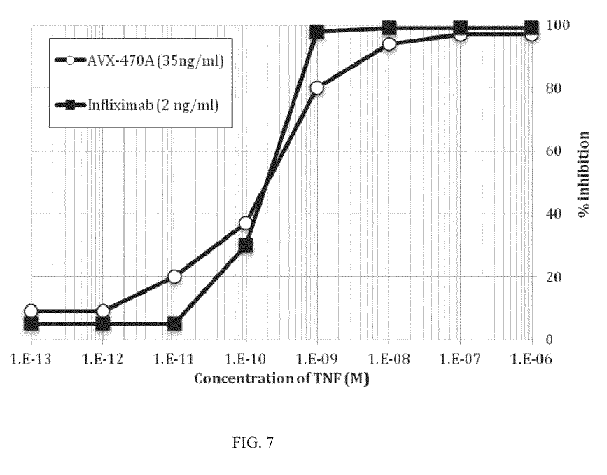

As shown in FIG. 7, both AVX-470A and infliximab had a measured affinity of 2×10⁻¹⁰ M. However, the shape of the titration curve was very different for the two agents. Infliximab, a monoclonal antibody that binds a single epitope on TNF, displayed a very sharp inhibition curve, with only a single point on the curve (1e-10M) falling between no binding and saturated binding. In contrast, AVX-470, a polyclonal antibody that binds multiple epitopes on TNF, displayed a much shallower inhibition curve, with 4 points on the curve (1e-11 to 1e-8) falling between to binding and saturated binding.

### Example 29

### Potency of Affinity-Purified AVX-470A as Evaluated by Binding in ELISA

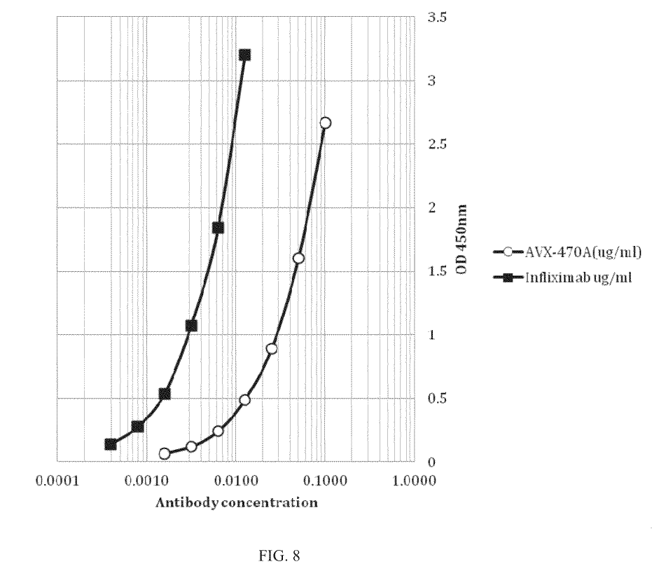

Affinity purified AVX-470 as prepared in Examples 3, 14 and 19(b) was assayed in a TNF-specific ELISA using the protocol described in Example 26(a). The humanized anti-TNF monoclonal antibody infliximab was used as a comparator. As shown in FIG. 8, the potency of AVX-470A was 10-fold lower than that of infliximab as defined by TNF binding as measured by ELISA.

### Example 30

### Potency of Affinity-Purified AVX-470A as Evaluated by Neutralization in L929 Assay

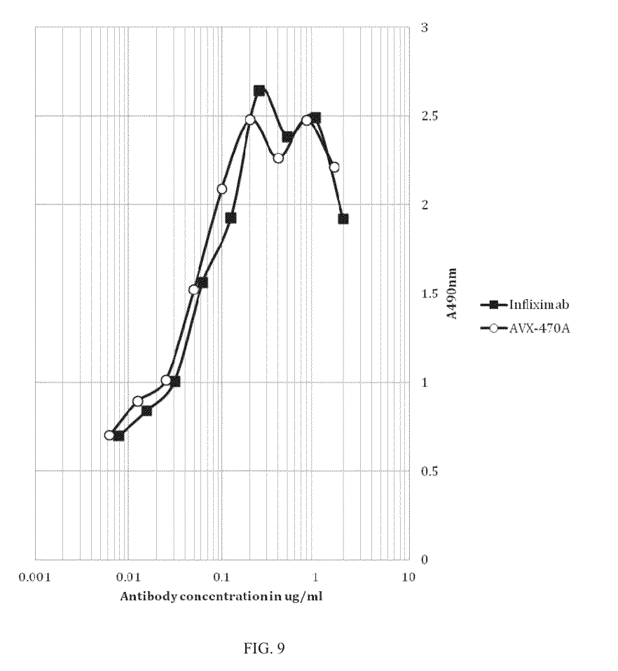

Affinity purified AVX-470 as prepared in Examples 3, 14 and 19(b) was assayed in a L929 assay to measure TNF neutralization using the protocol described in Example 26(b). The humanized anti-TNF monoclonal antibody infliximab was used as a comparator. As shown in FIG. 9, the potency of AVX-470A was 1.5-fold higher than that of infliximab as defined by TNF neutralization.

Together, Examples, 29 and 30 demonstrate that there is a lack of correlation between TNF binding as measured by ELISA and TNF neutralization as measured in the L929 assay. Although not intended to limit the scope of the invention by implying a mechanism, it may be that AVX-470 is more potent than would be expected based on binding data alone due to a synergistic effect of the polyclonal antibody binding to multiple epitopes on TNF.

### Example 31

### Induction of Apoptosis

Induction of apoptosis was carried out using an adaptation of protocols as described in Nesbitt, A. (2007) Inflamm. Bowel Dis. 13, 1323; Atreya, R. (2011) Gastroenter. 141, 2026; and Kaymakcalan, Z. (2006) Poster at FOCIS Annual Scientific Meeting. Briefly, human whole blood from healthy donors was purchased from Research Blood Components (Boston, Mass.). Whole blood was collected in BD Vacutainer CPT-Heparin tubes, which contain FICOLL Hypaque density fluid and a polyester gel barrier for separation of peripheral blood mononuclear cells (PBMC) by density centrifugation. PBMC were cultured at 37° C., 5% CO₂, and 95% humidity. IMDM medium was supplemented with 10% fetal calf serum, GlutaMAX (Gibco), penicillin and streptomycin. Cells were plated at 1×10⁶ cells/ml and stimulated with 5 ng/ml PMA and 1 μM Ionomycin for 48 hours. Cells were harvested, washed three times in complete medium, and replated at 1×10⁶ cells/ml alone, with 10 mg/ml AVX-470, or with 100 μg/ml Infliximab. Twenty-four hours later, cells were assessed for apoptosis by staining with annexin V and propidium iodide, and subjected to fluorescence activated cells sorting (FACS) analysis on a FACSCanto II (BD Biosciences).

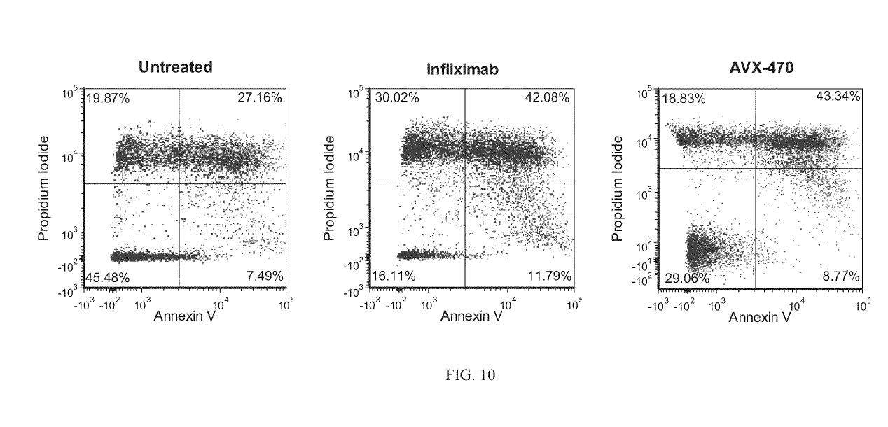

As shown in FIG. 10, cells expressing high levels of both propidium iodide and Annexin V (upper right quadrant) are late apoptotic cells while cells expressing high levels of Annexin V but low levels of propidium iodide (lower right quadrant) are early apoptotic cells. The data is summarized in Table 18.

TABLE 18

| Group | Late apoptotic cells | Total apoptotic cells |
|---|---|---|
| Untreated | 27.16% | 34.65% |
| Infliximab | 42.08% | 53.87% |
| AVX-470 | 43.34% | 52.11% |

Therefore, AVX-470 recognizes transmembrane TNF and induces apoptosis of transmembrane TNF-expressing cells. One mechanism implicated in apoptosis by an antibody binding transmembrane TNF is that the antibody induces reverse signaling through membrane-bound TNF and/or neutralizes the biological activity of transmembrane TNF such that antiapoptotic signaling is reduced thereby increasing apoptosis (Van den Brande et al. (2003) Gastroenterology 124:1774-1785).

### Example 32

### Assessment of Ability of AVX-470 to Bind Human IL-6

The binding of AVX-470 to rhTNF and rhIL-6 was evaluated in a sandwich ELISA format where the cytokine (TNF or IL-6) is captured by a plate-bound standard antibody using specific ELISA kits. Human TNF-alpha DuoSet Kit and Human Interleukin-6 Quantikine Kit were purchased from R&D Systems. The IL-6 kit comes with capture anti-IL-6 precoated and preblocked. For the TNF kit, the plate was coated with 4 ug/mL capture anti-TNF antibody and incubated overnight at room temperature, blocked for 1 hour with 1% BSA (bovine serum albumin) in PBS and washed. Two-fold dilutions of human TNF (1000-18.75 pg/ml) or IL-6 (300-3.12 pg/ml) standards were added and incubated for 2 hours at room temperature and the plates were again washed. The detection antibody from the kit (250 ng/ml), AVX-470 (1.5 mg/ml), control bovine Ig (1.5 mg/ml) or Infliximab (12.5 ng/ml) were added and incubated for 2 hours at room temperature and washed. Binding of AVX-470 was detected using HRP-labeled anti-bovine Ig; binding of infliximab was detected using HRP-labeled anti-human Ig; binding of the biotinylated standards provided with the kits was detected using HRP-labeled streptavidin. All plates were developed with TMB substrate. Data are expressed as absorbance at 450 nm with the background seen in the absence of cytokine subtracted from each value. The data is summarized in Table 19.

TABLE 19

Absorbance 450 nm

| | rhTNF | rhIL-6 |
|---|---|---|
| Test Antibody | (250 pg/ml) | (300 pg/ml) |
| Mouse anti-rhTNF Capture Ab | 1.59 | Not done |
| Mouse anti-rhIL-6 Capture Ab | Not done | 1.76 |
| AVX-470 1.5 mg/ml | 0.76 (pos) | 0.03 (neg) |
| Control Bovine Ig 1.5 mg/ml | −0.01 (neg) | 0.01 (neg) |
| Infliximab 12.5 ng/ml | 0.11 (pos) | −0.00 (neg) |

The data demonstrate that AVX-470 bound to recombinant human TNF but not to recombinant human IL-6. Control bovine immunoglobulin did not bind to either cytokine Infliximab bound to TNF but not to IL-6.

### Example 33

### Safety Testing of AVX-470 Bovine Anti-TNF Antibody

To evaluate the safety of AVX-470 in vivo, GLP toxicology studies in rats and cynomolgus monkeys were carried out at Calvert Laboratories, Inc. (Scott Township, Pa.). The purified antibody drug substance was administered by oral gavage twice per day for 28 days to male and female Sprague Dawley rats at a dosage of 250, 1000, and 4000 mg/kg/day (10 animals per sex per group). No unscheduled deaths occurred in any of the groups. No abnormal clinical observations were noted in any of the animals in the control saline group and AVX-470-low and mid and high dose groups. No test article-related effects on body weight were noted at any dose level. AVX-470 had no significant clinical pathology effects at any of the doses tested. There were no remarkable changes in clinical chemistry or hematology parameters in any of the dose groups. Lymphoid and gastrointestinal organs were examined microscopically from male and female Sprague Dawley rats euthanized on Day 29, from control/saline and AVX-470-high dose group. Under the conditions of this study, there were no AVX-470-related microscopic toxic effects on lymphoid and gastrointestinal organs at the highest dose level tested (4000 mg/kg/day) in Sprague Dawley rats.

AVX-470 was administered by oral gavage twice per day for 28 days to male and female cynomolgus monkeys (3 per sex per group) at a dosage of 125, 500, and 2000 mg/kg/day. No unscheduled deaths occurred in any of the groups. No remarkable clinical observations were noted in any of the animals in the control saline group and AVX-470-low and mid and high dose groups. No test article-related effects on body weight were noted at any dose level. AVX-470 had no significant clinical pathology effects at any of the doses tested. There were no remarkable changes in clinical chemistry or hematology parameters in any of the dose groups. Lymphoid and gastrointestinal organs were examined microscopically from male and female cynomolgus monkeys euthanized on Day 29. Under the conditions of this study, there were no AVX-470-related microscopic toxic effects on lymphoid and gastrointestinal organs at the highest dose level tested (2000 mg/kg/day) in cynomolgus monkeys.

The patent and scientific literature referred to herein establishes the knowledge that is available to those with skill in the art. All United States patents and published or unpublished United States patent applications cited herein are incorporated by reference.

All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A composition comprising polyclonal antibodies that specifically bind to human tumor necrosis alpha (hTNF) wherein the polyclonal antibodies are derived from the serum, milk or colostrum of a bovine animal that has been immunized with hTNF or an immunogenic portion thereof wherein the polyclonal antibodies comprise one or more of the following features:
a) neutralize the activity of human TNF and at least one non-human primate TNF selected from rhesus monkey TNF and cynomologus monkey TNF;
b) bind to at least one epitope on hTNF wherein at least one epitope comprises an amino acid sequence selected from all or a portion of the amino acid sequence of: SEQ ID NO: 2; SEQ ID NO: 3 SEQ ID NO: 4; SEQ ID NO: 5; and SEQ ID NO: 6; and
c) induce apoptosis in vitro in peripheral blood mononuclear cells expressing transmembrane TNF.

2. The composition of claim 1, wherein the polyclonal antibodies also bind and neutralize canine TNF.

3. The composition of claim 1, wherein the polyclonal antibodies have about 2% or less cross reactivity with murine TNF as compared to hTNF.

4. The composition of claim 1, wherein the polyclonal antibodies bind canine TNF to a greater degree than cynomolgus macaque TNF and neutralize cynomolgus macaque TNF to a greater degree than canine TNF.

5. The composition of claim 1, which neutralizes human TNF cytotoxicity in a standard in vitro L929 assay with an EC50 of 0.03 mg/ml or less.

6. The composition of claim 1, wherein the bovine animal is immunized with recombinant hTNF or an immunogenic fragment thereof.

7. The composition of claim 1, wherein the bovine animal is immunized with hTNF or an immunogenic fragment thereof in combination with an adjuvant selected from Quil A, Montanide ISA 201 VG, Montanide ISA-25, Emulsigen-D and Emulsigen-BCL.

8. A pharmaceutical composition comprising the composition of claim 1 and a pharmaceutically acceptable carrier or excipient.

9. The pharmaceutical composition of claim 8 further comprising at least one additional therapeutic agent.

10. A method of treating inflammatory bowel disease (IBD) in a patient comprising administering to the patient a therapeutically effective amount of the pharmaceutical composition of claim 8.

11. A method of treating oral or intestinal mucositis in a patient comprising administering to the patient a therapeutically effective amount of a composition of claim 8.

12. The method of claim 11, wherein the oral or intestinal mucositis is induced by chemotherapy or radiation therapy.

13. The method of claim 11, wherein the oral or intestinal mucositis is caused by non-therapeutic exposure to radiation.

14. A method of treating gastrointestinal acute radiation syndrome (GI-ARS) in a patient comprising administering to the patient a therapeutically effective amount of a composition of claim 8.

15. The method of claim 10, wherein the composition is administered orally or rectally.

16. A method of treating GI-ARS in a non-human animal model of GI-ARS comprising the steps of administering the composition of claim 8 to the non-human animal model for GI-ARS.

17. The method of claim 16 wherein the non-human animal model is selected from a non-human primate, a dog and a pig.

18. The method of claim 17, wherein the non-human primate is selected from a cynomolgus monkey and a rhesus monkey.

19. The composition of claim 1 which contains less than about 1 mg of lactoferrin per gram of total protein present in the composition.

20. The composition of claim 1, wherein the preparation of the composition comprises the steps of:
(a) filtering the whey derived from the colostrum of the bovine through an anion exchange column or a cationic exchange column;
(b) collecting the flow through of the column in step (a); and
(c) concentrating the flow through of step (b) by ultrafiltration.

21. The composition of claim 1, wherein the preparation of the composition comprises the steps of:
(a) adjusting the pH of whey derived from the colostrum of the bovine to a pH of 6.6 to 7.0;
(b) filtering the whey through an anion exchange column connected in series with a cation exchange column wherein the whey sequentially flows through both columns connected in series without addition of materials that change the salt concentration or pH;
(c) collecting the flow through after it passes through both columns of step (b) without addition of materials that change the salt concentration or pH before collection occurs; and
(d) concentrating the flow through of step (b) by ultrafiltration.

22. The composition of claim 21, wherein the specific activity of the polyclonal antibodies present in the whey is increased by about 2 fold in the concentrated flow through of step (d).

23. The composition of claim 21, wherein the preparation of the composition further comprises step (e) affinity purifying the concentrate of step (d) using an affinity matrix coupled to hTNF.

24. The composition of claim 23, wherein the neutralizing activity of hTNF cytotoxicity as measured in a standard in vitro L929 assay of the affinity purified material of step (e) is increased by at least 100 fold as compared to the neutralizing activity of the concentrated flow through of step (d).


SEQUENCE LISTING (9 sequences)

SEQ ID NO: 1 - protein, 157 aa, Homo sapien [sequence omitted]

SEQ ID NO: 2 - protein, 15 aa, Homo sapien
VRSSSRTPSDKPVAH

SEQ ID NO: 3 - protein, 15 aa, Homo sapien
QAEGQLQWLNRRANA

SEQ ID NO: 4 - protein, 5 aa, Homo sapien
QVLFK

SEQ ID NO: 5 - protein, 5 aa, Homo sapien
VNLLS

SEQ ID NO: 6 - protein, 10 aa, Homo sapien
RLSAEINRPD

SEQ ID NO: 7 - protein, 15 aa, Canine
VKSSSRTPSDKPVAH

SEQ ID NO: 8 - protein, 15 aa, Feline
LRSSSRTPSDKPVAH

SEQ ID NO: 9 - protein, 12 aa, Homo sapien
SSRTPSDKPVAH